SEQUENCE LISTING (1 sequence)

SEQ ID NO: 1 - DNA, 21 bp, Homo sapiens
cagcagcagagtcttcatcat

(19) United States
(12) Patent Application Publication
(10) Pub. No.: US 2013/0017272 A1
(43) Pub. Date: Jan. 17, 2013

(54) METHOD FOR TREATING NON-SMALL CELL LUNG CANCER

(71) Applicants: Chen DUKSIN, Tel Aviv (IL); Shoshi Tessler, Zichron Ya'acov (IL)

(72) Inventors: Chen DUKSIN, Tel Aviv (IL); Shoshi Tessler, Zichron Ya'acov (IL)

(21) Appl. No.: 13/475,780

(22) Filed: May 18, 2012

Related U.S. Application Data
(60) Provisional application No. 61/487,918, filed on May 19, 2011.
(60) Provisional application No. 61/493,346, filed on Jun. 3, 2011.

(51) Int. Cl. A61K 31/711; A61P 35/04; A61K 33/24; A61P 35/00

(52) U.S. Cl. 424/649; 514/44 A

(57) ABSTRACT

A method of treating a human patient afflicted with lung cancer comprising periodically administering to the human patient chemotherapy comprising an amount of a taxane and 640 mg of an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, thereby treating the human patient afflicted with cell lung cancer.

This application claims the benefit of U.S. Provisional Application No. 61/487,918, filed May 19, 2011 and U.S. Provisional Application No. 61/493,346, Jun. 3, 2011, the contents of which are hereby incorporated by reference.

Throughout this application, various publications are referenced, including referenced in parenthesis. Full citations for publications referenced in parenthesis may be found listed in alphabetical order at the end of the specification immediately preceding the claims. The disclosures of all referenced publications in their entireties are hereby incorporated by reference into this application in order to more fully describe the state of the art to which this invention pertains.

### REFERENCE TO SEQUENCE LISTING

This application incorporates-by-reference nucleotide and/or amino acid sequences which are present in the file named “120518_{—}2609_{—}82439 A Sequence Listing GC.txt,” which is 452 bytes in size, and which was created May 18, 2012 in the IBM-PC machine format, having an operating system compatibility with MS-Windows, which is contained in the text file filed May 18, 2012 as part of this application.

### BACKGROUND OF INVENTION

Lung cancer was the most commonly diagnosed cancer as well as a leading cause of cancer death in males in 2008 globally. Among females, it was the fourth most commonly diagnosed cancer and the second leading cause of cancer death. Worldwide, lung cancer accounted for 13% (1.6 million) of the total cases and 18% (1.4 million) of the cancer deaths in 2008. The majority of lung neoplasms are non-small cell lung cancers (NSCLC) (Jemal et al., 2011; D'Addario et al., 2010). First-line chemotherapy regimens for NSCLC often comprise the platinum doublet, which means adding a second chemotherapy drug (paclitaxel, pemetrexed, gemcitabine, vinorelbine, etc.) to a platinum based drug (cisplatin or carboplatin) (D'Addario et al., 2010; National Comprehensive Cancer Network Clinical Practice Guidelines in Oncology, Non-Small Cell Lung Cancer, V.2.2010). Reported median survival among these doublets does not differ dramatically, and is in the range of approximately 8-10 months (D'Addario et al., 2010; National Comprehensive Cancer Network Clinical Practice Guidelines in Oncology, Non-Small Cell Lung Cancer, V.2.2010). Despite the availability of several active chemotherapeutic agents, long-term survival rates remain <15% in these patients (D'Addario et al., 2010; National Comprehensive Cancer Network Clinical Practice Guidelines in Oncology, Non-Small Cell Lung Cancer, V.2.2010). Therefore, treatments that significantly prolong the survival of patients afflicted with NSCLC are needed.

Clusterin is a secretable cytoprotective protein that is upregulated in response to a number of tumor cell killing interventions, specifically chemotherapy, hormone ablation therapy and radiation therapy. As described in U.S. Patent Application Publication No. 2008/0119425, the contents of which are incorporated herein by reference, clusterin is expressed in many malignancies including NSCLC, as well as prostate cancer, bladder cancer, ovarian cancer, renal cancer, melanoma, and pancreatic cancer.

Custirsen is a second-generation antisense oligonucleotide that inhibits clusterin expression. Custirsen is designed specifically to bind to a portion of clusterin mRNA, resulting in the inhibition of the production of clusterin protein. The structure of custirsen is available, for example, in U.S. Pat. No. 6,900,187, the contents of which are incorporated herein by reference. A broad range of studies have shown that custirsen potently reduces the expression of clusterin, facilitates apoptosis, and sensitizes cancerous human prostate, breast, ovarian, lung, renal, bladder, and melanoma cells to chemotherapy (Miyake et al. 2005), see also, U.S. Patent Application Publication No. 2008/0119425 A1, the contents of which are incorporated herein by reference.

### Paclitaxel, Docetaxel and Carboplatin

Paclitaxel and docetaxel are mitotic inhibitors that are used as chemotherapeutic agents in the treatment of cancer (Rowinsky et al., 1990). They belong to a class of drugs called taxanes, and act by stabilizing microtubules, thus disrupting their function during cell division (Kuriyama, 1986; Rowinsky et al., 1990).

Carboplatin is an alkylating agent that acts by interacting with DNA, which interferes with cellular repair mechanisms, ultimately resulting in cell death (Knox et al., 1986; Teicher et al., 1989). Carboplatin belongs to a class of drugs called platinum-based chemotherapeutics.

### Combination Therapy

Clinical studies have described the combination of carboplatin/paclitaxel with agents such as bevacizumab or cetuximab for the treatment of NSCLC (Sandler et al., 2006; Pirker et al., 2009); however, treatment of NSCLC with a combination of carboplatin/paclitaxel and an antisense oligonucleotide has not been attempted. Furthermore, such a combination has not been described for the treatment of populations consisting of patients with Stage IV NSCLC or NSCLC of non-squamous histology.

The administration of multiple drugs to treat a given condition, such as NSCLC, raises a number of potential problems. In vivo interactions between multiple drugs are complex. The effects of any single drug are related to its absorption, distribution, and elimination. When multiple drugs are introduced into the body, each drug can affect the absorption, distribution, and elimination of the other and hence, alter the effects of the other. For instance, one drug may inhibit, activate or induce the production of enzymes involved in a metabolic route of elimination of another drug (Guidance for Industry, 1999). Thus, when two drugs are administered to treat the same condition, it is unpredictable whether each will complement, have no effect on, or interfere with, the therapeutic activity of the other in a human patient.

Not only may the interaction between multiple drugs affect the intended therapeutic activity of each drug, but the interaction may increase the levels of toxic metabolites (Guidance for Industry, 1999). The interaction may also heighten or lessen the side effects of each drug. Hence, upon administration of two drugs to treat a disease, it is unpredictable what change will occur in the negative side profile of each drug.

Additionally, it is difficult to accurately predict when the effects of the interaction between the multiple drugs will become manifest. For example, metabolic interactions between drugs may become apparent upon the initial administration of the second drug, after the two have reached a steady-state concentration or upon discontinuation of one of the drugs (Guidance for Industry, 1999).

Thus, the success of one drug or each drug alone in an in vitro model, an animal model, or in humans, may not correlate into efficacy of the administration of a combination of the drugs together.

### SUMMARY OF THE INVENTION

The present invention provides a method of treating a human patient afflicted with unresectable, advanced or metastatic non-small cell lung cancer comprising periodically administering to the human patient chemotherapy comprising an amount of a taxane, and 640 mg of an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, thereby treating the human patient afflicted with unresectable, advanced or metastatic non-small cell lung cancer.

The present invention also provides a combination for treating a human patient afflicted with unresectable, advanced or metastatic non-small cell lung cancer, comprising chemotherapy comprising a taxane and an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19. In some embodiments, the combination is for treating a human patient afflicted with non-small cell lung cancer of non-squamous histology or Stage IV non-small cell lung cancer.

The present invention also provides a composition for treating a human patient afflicted with unresectable, advanced or metastatic non-small cell lung cancer, comprising chemotherapy consisting of a taxane and, optionally, a platinum-based chemotherapeutic agent; and an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19. In some embodiments, the composition is for treating a human patient afflicted with non-small cell lung cancer of non-squamous histology or Stage IV non-small cell lung cancer.

The present invention also provides a pharmaceutical composition for treating a human patient afflicted with unresectable, advanced or metastatic non-small cell lung cancer, the composition comprising chemotherapy comprising a taxane and, optionally, a platinum-based chemotherapeutic agent, and an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19. In some embodiments, the pharmaceutical composition is for treating a human patient afflicted with non-small cell lung cancer of non-squamous histology or Stage IV non-small cell lung cancer.

Some embodiments of the present invention relate to the use of a composition comprising chemotherapy comprising a taxane and, optionally, a platinum-based chemotherapeutic agent, and an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, for treatment of a human patient afflicted with unresectable, advanced or metastatic non-small cell lung cancer. In some embodiments, the use of the composition is for the treatment of a human patient afflicted with non-small cell lung cancer of non-squamous histology or Stage IV non-small cell lung cancer.

Some embodiments of the present invention relate to the use of a composition comprising chemotherapy comprising a taxane and, optionally, a platinum-based chemotherapeutic agent, and an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, for preparation of a medicament for treatment of a human patient afflicted with unresectable, advanced or metastatic non-small cell lung cancer. In some embodiments, the use of the composition is for preparation of a medicament for treatment of a human patient afflicted with unresectable, advanced or metastatic non-small cell lung cancer. In some embodiments, the use of the composition is for preparation of a medicament for treatment of a human patient afflicted with non-small cell lung cancer of non-squamous histology or Stage IV non-small cell lung cancer.

The present invention also provides a package for use in the treatment of a human patient afflicted with unresectable, advanced or metastatic non-small cell lung cancer, comprising chemotherapy comprising a taxane and, optionally, a platinum-based chemotherapeutic agent, and an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, and instructions for the use of the chemotherapy in combination with the anti-clusterin oligonucleotide for the treatment of unresectable, advanced or metastatic non-small cell lung cancer. In some embodiments, the package is for use in the treatment of a human patient afflicted with non-small cell lung cancer of non-squamous histology or Stage IV non-small cell lung cancer.

The present invention also provides a chemotherapy comprising a taxane and, optionally, a platinum-based chemotherapeutic agent, for use in combination with an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, for treating of a human patient afflicted with unresectable, advanced or metastatic non-small cell lung cancer; or an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, for use in combination with a chemotherapy comprising a taxane and, optionally, a platinum-based chemotherapeutic agent, for treating of a human patient afflicted with unresectable, advanced or metastatic non-small cell lung cancer. In some embodiments, the chemotherapy in combination with the anti-clusterin oligonucleotide is for treating a human patient afflicted with non-small cell lung cancer of non-squamous histology or Stage IV non-small cell lung cancer. In some embodiments, the anti-clusterin oligonucleotide in combination with the chemotherapy is for treating a human patient afflicted with non-small cell lung cancer of non-squamous histology or Stage IV non-small cell lung cancer.

The present invention also provides a method of treating a human patient afflicted with non-small cell lung cancer of non-squamous histology or Stage IV non-small cell lung cancer comprising periodically administering to the human patient chemotherapy consisting of an amount of paclitaxel and an amount of carboplatin; and 640 mg of an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, thereby treating the human patient afflicted with non-small cell lung cancer of non-squamous histology or Stage IV non-small cell lung cancer.

Some embodiments of the present invention provide a combination for treating a human patient afflicted with non-small cell lung cancer of non-squamous histology or Stage IV non-small cell lung cancer, comprising chemotherapy consisting of paclitaxel and carboplatin, and an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19.

Some embodiments of the present invention provide a composition for treating a human patient afflicted with non-small cell lung cancer of non-squamous histology or Stage IV non-small cell lung cancer, comprising chemotherapy consisting of paclitaxel and carboplatin, and an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19.

Some embodiments of the present invention provide a pharmaceutical composition for treating a human patient afflicted with non-small cell lung cancer of non-squamous histology or Stage IV non-small cell lung cancer, the composition comprising chemotherapy consisting of paclitaxel and carboplatin, and an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19.

Some embodiments of the present invention relate to the use of a composition comprising chemotherapy consisting of paclitaxel and carboplatin, and an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, for treatment of a human patient afflicted with non-small cell lung cancer of non-squamous histology or Stage IV non-small cell lung cancer.

Some embodiments of the present invention relate to the use of a composition comprising chemotherapy consisting of paclitaxel and carboplatin, and an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, for preparation of a medicament for treatment of a human patient afflicted with non-small cell lung cancer of non-squamous histology or Stage IV non-small cell lung cancer.

Some embodiments of the present invention provide a package for use in the treatment of a human patient afflicted with non-small cell lung cancer of non-squamous histology or Stage IV non-small cell lung cancer, comprising chemotherapy consisting of paclitaxel and carboplatin, and an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, and instructions for the use of the chemotherapy in combination with the anti-clusterin oligonucleotide for the treatment of non-small cell lung cancer of non-squamous histology or Stage IV non-small cell lung cancer.

Some embodiments of the present invention provide a chemotherapy consisting of paclitaxel and carboplatin, for use in combination with an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, for treating of a human patient afflicted with non-small cell lung cancer of non-squamous histology or Stage IV non-small cell lung cancer; or an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, for use in combination with a chemotherapy consisting of paclitaxel and carboplatin, for treating of a human patient afflicted with non-small cell lung cancer of non-squamous histology or Stage IV non-small cell lung cancer.

The present invention also provides a method of treating a human patient afflicted with unresectable, advanced or metastatic non-small cell lung cancer comprising periodically administering to the human patient chemotherapy comprising an amount of docetaxel; and 640 mg of an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, thereby treating the human patient afflicted with unresectable, advanced or metastatic non-small cell lung cancer.

The present invention also provides a combination for treating a human patient afflicted with unresectable, advanced or metastatic non-small cell lung cancer, comprising chemotherapy comprising docetaxel; and an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19. In some embodiments, the combination is for treating a human patient afflicted with non-small cell lung cancer of non-squamous histology or Stage IV non-small cell lung cancer.

The present invention also provides a composition for treating a human patient afflicted with unresectable, advanced or metastatic non-small cell lung cancer, comprising chemotherapy comprising docetaxel; and an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19. In some embodiments, the composition is for treating a human patient afflicted with non-small cell lung cancer of non-squamous histology or Stage IV non-small cell lung cancer.

The present invention also provides a pharmaceutical composition for treating a human patient afflicted with unresectable, advanced or metastatic non-small cell lung cancer, the composition comprising chemotherapy comprising docetaxel; and an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19. In some embodiments, the pharmaceutical composition is for treating a human patient afflicted with non-small cell lung cancer of non-squamous histology or Stage IV non-small cell lung cancer.

Some embodiments of the present invention relate to the use of a composition comprising chemotherapy comprising docetaxel; and an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, for treatment of a human patient afflicted with unresectable, advanced or metastatic non-small cell lung cancer. In some embodiments, the use of the composition is for treatment of a human patient afflicted with non-small cell lung cancer of non-squamous histology or Stage IV non-small cell lung cancer.

Some embodiments of the present invention relate to the use of a composition comprising chemotherapy comprising docetaxel; and an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, for preparation of a medicament for treatment of a human patient afflicted with unresectable, advanced or metastatic non-small cell lung cancer. In some embodiments, the use of the composition is for preparation of a medicament for treatment of a human patient afflicted with non-small cell lung cancer of non-squamous histology or Stage IV non-small cell lung cancer.

The present invention also provides a package for use in the treatment of a human patient afflicted with unresectable, advanced or metastatic non-small cell lung cancer, comprising chemotherapy comprising docetaxel; and an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, and instructions for the use of the chemotherapy in combination with the anti-clusterin oligonucleotide for the treatment of unresectable, advanced or metastatic non-small cell lung cancer. In some embodiments, the package is for use in the treatment of a human patient afflicted with non-small cell lung cancer of non-squamous histology or Stage IV non-small cell lung cancer.

The present invention also provides a chemotherapy comprising docetaxel for use in combination with an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, for treating of a human patient afflicted with unresectable, advanced or metastatic non-small cell lung cancer; or an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, for use in combination with a chemotherapy comprising docetaxel, for treating of a human patient afflicted with unresectable, advanced or metastatic non-small cell lung cancer. In some embodiments, the chemotherapy in combination with the anti-clusterin oligonucleotide is for treating a human patient afflicted with non-small cell lung cancer of non-squamous histology or Stage IV non-small cell lung cancer. In some embodiments, the anti-clusterin oligonucleotide in combination with the chemotherapy is for treating a human patient afflicted with non-small cell lung cancer of non-squamous histology or Stage IV non-small cell lung cancer.

### BRIEF DESCRIPTION OF THE DRAWINGS

FIG. 1. Treatment Design for the Combination of Custirsen and Paclitaxel/Carboplatin.

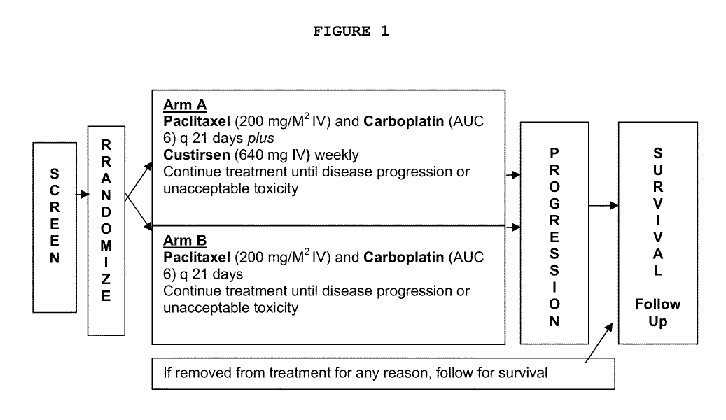

FIG. 2. Study Timeline for Clinical Trial Evaluating the Safety and Efficacy of the Combination of Custirsen and Paclitaxel/Carboplatin or the Combination of Custirsen and Docetaxel, for the treatment of NSCLC.

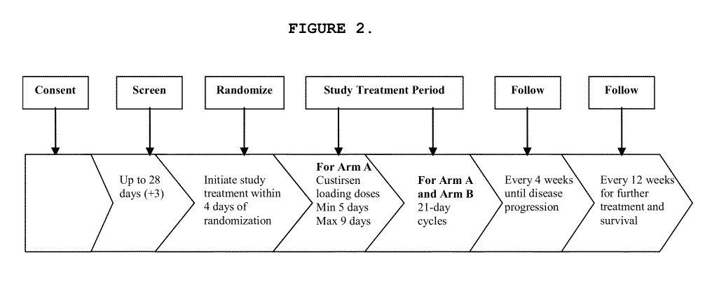

FIG. 3. Treatment Scheme for Clinical Trial Evaluating the Safety and Efficacy of the Combination of Custirsen and Paclitaxel/Carboplatin for the treatment of Stage IV NSCLC of Non-squamous Histology.

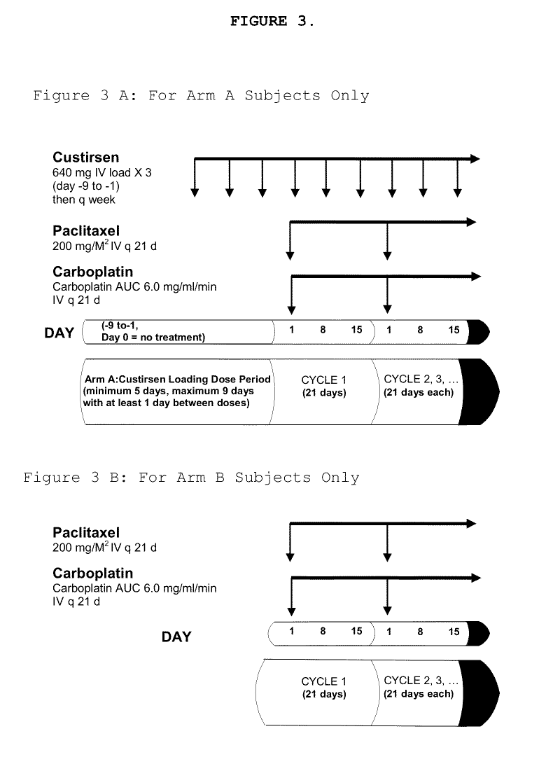

FIG. 4. Survival Curves for Low vs. High Baseline Clusterin in patients with NSCLC. Survival Curves for Low vs. High Baseline Clusterin. The Figure shows Kaplan-Meier survival curves for the N=55 subjects with at least one post-baseline clusterin assessment. The subjects were stratified by their baseline clusterin level: Low (≦71 μg/mL) vs. High (>71 μg/mL). The log-rank test gave p=0.0002.

FIG. 5. Kaplan-Meier curves corresponding to the 71 μg/mL cutpoint for baseline clusterin and a 33 μg/mL cutpoint for average clusterin in patients with NSCLC. The Figure shows Kaplan-Meier survival curves for N=54 of the N=55 subjects with both baseline and post-baseline clusterin assessments. The subjects were stratified by their baseline clusterin level (≦71 μg/mL vs. >71 μg/mL), and also by AUCp, the time-weighted average of their post-baseline clusterin levels (≦33 μg/mL vs. >33 μg/mL). The log-rank test comparing the three curves gave p=0.0003. (Please see Example 2 regarding the missing subject.)

FIG. 6. Kaplan-Meier curves corresponding to the 71 μg/mL cutpoint for baseline clusterin and a 30 μg/mL cutpoint for minimum clusterin. The figure shows Kaplan-Meier survival curves for N=53 of the N=55 subjects with both baseline and post-baseline clusterin assessments. The subjects were stratified by their baseline clusterin level (≦71 μg/mL vs. >71 μg/mL), and also by their minimum on-study clusterin levels (≦30 μg/mL vs. >30 μg/mL). The log-rank test comparing the three curves gave p=0.0002. (Please see Example 2 regarding the two missing subjects.)

FIG. 7. Treatment Design for the Combination of Custirsen and Docetaxel.

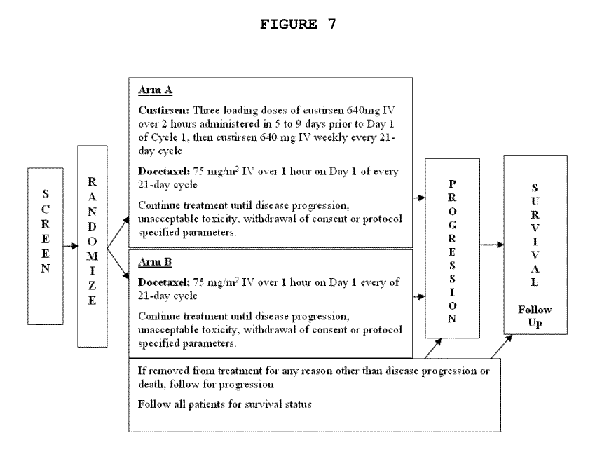

### DETAILED DESCRIPTION OF THE INVENTION

The present invention describes novel methods and compositions effective for the treatment of lung cancer. In some embodiments, the present invention describes novel methods and compositions effective for the treatment of certain types of NSCLC, including, unresectable, advanced or metastatic (Stage IV per AJCC 7^{th} edition TNM staging) NSCLC and NSCLCL of non-squamous histology and Stage IV NSCLC.

The present invention provides a method of treating a human patient afflicted with unresectable, advanced or metastatic non-small cell lung cancer comprising periodically administering to the human patient chemotherapy comprising an amount of a taxane, and 640 mg of an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, thereby treating the human patient afflicted with unresectable, advanced or metastatic non-small cell lung cancer.

The present invention provides a method of treating a human patient afflicted with unresectable, advanced or metastatic (Stage IV per AJCC 7^{th} edition TNM staging) non-small cell lung cancer comprising periodically administering to the human patient chemotherapy comprising an amount of a taxane, and 640 mg of an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, thereby treating the human patient afflicted with unresectable, advanced or metastatic (Stage IV per AJCC 7^{th} edition TNM staging) non-small cell lung cancer.

Some embodiments of the present invention provide a method of treating a human patient afflicted with non-small cell lung cancer of non-squamous histology or Stage IV non-small cell lung cancer comprising periodically administering to the human patient chemotherapy comprising an amount of a taxane, and 640 mg of an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, thereby treating the human patient afflicted with non-small cell lung cancer of non-squamous histology or Stage IV non-small cell lung cancer.

In some embodiments, the taxane is paclitaxel.

In some embodiments, during the chemotherapy the amount of paclitaxel administered is 200 mg/m² intravenously to the human patient over a period of 3 hours.

In some embodiments, during the chemotherapy the amount of paclitaxel administered is less than 200 mg/m² intravenously to the human patient.

In some embodiments, during the chemotherapy the paclitaxel is administered to the human patient on the first day of each of up to six three-week chemotherapy cycles.

In some embodiments, the taxane is other than paclitaxel.

In some embodiments, the taxane is docetaxel, baccatin III, baccatin V, taxol B (cephalomannine), taxol C, taxol D, taxol E, taxol F, taxol G, cabazitaxel, larotaxel, ortataxel (14 beta-hydroxydeacetyl baccatin III), tesetaxol, 10-deacetyl baccatin III, 7-xylosyl-10-deacetyl cephalomannine, 7-xylosyl-10-deacetyl paclitaxel, 10-deacetyl cephalomannine, 7-xylosyl-10-deacetyl taxol C, 10-deacetyl paclitaxel, 7-xylosyl paclitaxel, 10-deacetyl taxol C, 10-deacetyl-7-epi cephalomaunine, 7-xylosyl taxol C, 10-deacetyl-7-epipaclitaxel, 7-epi cephalomaunine, 7-epi paclitaxel, 7-O-methylthiomethyl paclitaxel, 7-deoxy docetaxel, taxanime M, PG-paclitaxel, or DHA-paclitaxel.

In some embodiments, the taxane is docetaxel.

In some embodiments, the chemotherapy the amount of docetaxel administered is 75 mg/m² intravenously to the human patient over a period of 1 hour.

In some embodiments, the chemotherapy the amount of docetaxel administered is less than 75 mg/m² intravenously to the human patient.

In some embodiments, during the chemotherapy the docetaxel is administered to the human patient on the first day of each three-week chemotherapy cycle.

In some embodiments, the taxane is cabazitaxel.

In some embodiments, the chemotherapy further comprises an amount of a platinum-based chemotherapeutic agent.

In some embodiments, the platinum-based chemotherapeutic agent is cisplatin, carboplatin (paraplatin), nedaplatin, oxaliplatin, triplatin tetranitrate, satraplatin, iproplatin, lobaplatin, or picoplatin.

In some embodiments, the platinum-based chemotherapeutic agent is carboplatin.

In some embodiments, during the chemotherapy the amount of carboplatin administered is AUC 6 mg/mL/min intravenously to the human patient over a period of 30 minutes.

In some embodiments, during the chemotherapy the amount of carboplatin administered is less than AUC 6 mg/mL/min intravenously to the human patient over a period of 30 minutes.

In some embodiments, during the chemotherapy the carboplatin is administered to the human patient on the first day of each of up to six three-week chemotherapy cycles.

In some embodiments, the platinum-based chemotherapeutic agent is cisplatin.

In some embodiments, the platinum-based chemotherapeutic agent is other than carboplatin.

In some embodiments, during the chemotherapy the platinum-based chemotherapeutic agent is administered to the human patient on the first day of each three-week chemotherapy cycle.

In some embodiments, during the chemotherapy the taxane is administered to the human patient on the first day of each three-week chemotherapy cycle.

In some embodiments, the non-small cell lung cancer is stage IV lung cancer.

In some embodiments, the non-small cell lung cancer is of non-squamous histology.

In some embodiments, the treating includes prolonging survival of the human patient.

In some embodiments, the treating includes prolonging survival of the human patient which prolonged survival is free of progression of the non-small lung cancer.

In some embodiments, the human patient survives free of progression of the lung cancer for at least 14 weeks.

In some embodiments, the human patient survives free of progression of the non-small cell lung cancer for at least 14 weeks.

In some embodiments, the human patient survives free of progression of the non-small cell lung cancer of non-squamous histology for at least 14 weeks.

In some embodiments, the human patient suffers from chest pain, pleural effusions, pulmonary edema, dyspnea, or hemoptysis.

In some embodiments, the lung cancer is lung adenocarcinoma or lung large cell carcinoma.

In some embodiments, the non-small cell lung cancer is lung adenocarcinoma or lung large cell carcinoma.

In some embodiments, the non-small cell lung cancer of non-squamous histology is lung adenocarcinoma or lung large cell carcinoma.

In some embodiments, the anti-clusterin oligonucleotide is administered to the human patient intravenously in an aqueous solution comprising sodium ions.

In some embodiments, the anti-clusterin oligonucleotide is administered to the human patient 3 times within a 5 to 9 day period before the first day of chemotherapy and then once weekly beginning on the first day of chemotherapy.

In some embodiments, the lung cancer is nonresectable, advanced or metastatic non-small cell lung cancer.

In some embodiments, the lung cancer has been histologically or cytologically confirmed and is, unresectable, advanced or metastatic (Stage IV per AJCC 7^{th} edition TNM staging).

In some embodiments, the lung cancer is Stage IV disease (according to the IASLC 7^{th} edition TNM staging, including subjects with pleural effusion who were previously classified as Stage IIIB) that is not amenable to either surgery or radiation therapy of curative intent.

In some embodiments, the human patient has not received treatment for non-small cell lung cancer for at least 1 year.

In some embodiments, the human patient has not received a chemotherapeutic agent for the treatment of non-small cell lung cancer for at least 1 year.

In some embodiments, the human patient has before initiation of the periodic administration received a chemotherapeutic agent for the treatment of lung cancer.

In some embodiments, the chemotherapeutic agent was a platinum-based chemotherapeutic agent.

In some embodiments, a method of the invention for treating a human patient afflicted with non-small cell lung cancer of non-squamous histology or Stage IV non-small cell lung cancer further comprises the steps of:
i) measuring the level of serum clusterin present in the blood of the human patient prior to the administration of the anti-clusterin oligonucleotide;
ii) determining whether the level of serum clusterin present in the human patient is lower than a predetermined upper threshold level of baseline serum clusterin below which a human patient is likely to substantially benefit from anti-clusterin therapy; and
iii) administering the anti-clusterin oligonucleotide only if the level of serum clusterin present in the blood of the human patient is lower than the predetermined upper threshold level of baseline serum clusterin.

In some embodiments, in step i) the measuring is performed after initiation of the chemotherapy.

In some embodiments, the predetermined upper threshold level of baseline serum clusterin is 75 μg/mL.

In some embodiments, a method of the invention for treating a human patient afflicted with non-small cell lung cancer of non-squamous histology or Stage IV non-small cell lung cancer further comprises the steps of:
i) administering to the human patient the anti-clusterin oligonucleotide in an initial dosage and treatment protocol;
ii) thereafter testing the human patient to determine a level of serum clusterin after a period of treatment with the anti-clusterin oligonucleotide intended to reduce clusterin expression;
iii) determining an adjusted dosage and treatment protocol based on the determined level of serum clusterin; and
iv) administering to the human patient the anti-clusterin oligonucleotide in accordance with the adjusted dosage and treatment protocol.

In some embodiments, the determined level of serum clusterin after a period of treatment with the anti-clusterin oligonucleotide intended to reduce clusterin expression is above a predetermined post anti-clusterin oligonucleotide initiation threshold level.

In some embodiments, the predetermined post anti-clusterin oligonucleotide initiation threshold level is 30 μg/mL.

In some embodiments, the adjusted dosage and treatment protocol comprises administration of the anti-clusterin oligonucleotide to the human patient two or three times per week.

The present invention also provides a combination for treating a human patient afflicted with unresectable, advanced or metastatic non-small cell lung cancer, comprising chemotherapy comprising a taxane and an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19. In some embodiments, the combination is for treating a human patient afflicted with non-small cell lung cancer of non-squamous histology or Stage IV non-small cell lung cancer.

The present invention also provides a composition for treating a human patient afflicted with unresectable, advanced or metastatic non-small cell lung cancer, comprising chemotherapy consisting of a taxane and, optionally, a platinum-based chemotherapeutic agent; and an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19. In some embodiments, the composition is for treating a human patient afflicted with non-small cell lung cancer of non-squamous histology or Stage IV non-small cell lung cancer.

The present invention also provides a pharmaceutical composition for treating a human patient afflicted with unresectable, advanced or metastatic non-small cell lung cancer, the composition comprising chemotherapy comprising a taxane and, optionally, a platinum-based chemotherapeutic agent, and an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19. In some embodiments, the pharmaceutical composition is for treating a human patient afflicted with non-small cell lung cancer of non-squamous histology or Stage IV non-small cell lung cancer.

Some embodiments of the present invention relate to the use of a composition comprising chemotherapy comprising a taxane and, optionally, a platinum-based chemotherapeutic agent, and an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, for treatment of a human patient afflicted with unresectable, advanced or metastatic non-small cell lung cancer. In some embodiments, the use of the composition is for treating a human patient afflicted with non-small cell lung cancer of non-squamous histology or Stage IV non-small cell lung cancer.

Some embodiments of the present invention relate to the use of a composition comprising chemotherapy comprising a taxane and, optionally, a platinum-based chemotherapeutic agent, and an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, for preparation of a medicament for treatment of a human patient afflicted with unresectable, advanced or metastatic non-small cell lung cancer. In some embodiments, the use of the composition is for preparation of a medicament for treatment of a human patient afflicted with unresectable, advanced or metastatic non-small cell lung cancer. In some embodiments, the use of the composition is for preparation of a medicament for treatment of a human patient afflicted with non-small cell lung cancer of non-squamous histology or Stage IV non-small cell lung cancer.

The present invention also provides a package for use in the treatment of a human patient afflicted with unresectable, advanced or metastatic non-small cell lung cancer, comprising chemotherapy comprising a taxane and, optionally, a platinum-based chemotherapeutic agent, and an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, and instructions for the use of the chemotherapy in combination with the anti-clusterin oligonucleotide for the treatment of unresectable, advanced or metastatic non-small cell lung cancer. In some embodiments, the package is for use in the treatment of a human patient afflicted with non-small cell lung cancer of non-squamous histology or Stage IV non-small cell lung cancer.

The present invention also provides a chemotherapy comprising a taxane and, optionally, a platinum-based chemotherapeutic agent, for use in combination with an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, for treating of a human patient afflicted with unresectable, advanced or metastatic non-small cell lung cancer; or an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, for use in combination with a chemotherapy comprising a taxane and, optionally, a platinum-based chemotherapeutic agent, for treating of a human patient afflicted with unresectable, advanced or metastatic non-small cell lung cancer. In some embodiments, the chemotherapy in combination with the anti-clusterin oligonucleotide is for treating a human patient afflicted with non-small cell lung cancer of non-squamous histology or Stage IV non-small cell lung cancer. In some embodiments, the anti-clusterin oligonucleotide in combination with the chemotherapy is for treating a human patient afflicted with non-small cell lung cancer of non-squamous histology or Stage IV non-small cell lung cancer.

The present invention provides a method of treating a human patient afflicted with non-small cell lung cancer of non-squamous histology or Stage IV non-small cell lung cancer comprising periodically administering to the human patient chemotherapy consisting of an amount of paclitaxel and an amount of carboplatin; and 640 mg of an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, thereby treating the human patient afflicted with non-small cell lung cancer of non-squamous histology or Stage IV non-small cell lung cancer.

In some embodiments, the treating includes prolonging survival of the human patient.

In some embodiments, the treating includes prolonging survival of the human patient which prolonged survival is free of progression of the non-small cell lung cancer.

In some embodiments, the human patient survives with a lower rate of progression of the non-small cell lung cancer of non-squamous histology for at least 14 weeks.

In some embodiments, the human patient survives free of progression of the non-small cell lung cancer of non-squamous histology for at least 8 weeks.

In some embodiments, the human patient survives free of progression of the non-small cell lung cancer of non-squamous histology for at least 14 weeks.

In some embodiments, the human patient survives free of progression of the non-small cell lung cancer of non-squamous histology for at least 20 weeks.

In some embodiments, the human patient survives free of progression of the non-small cell lung cancer of non-squamous histology for at least 26 weeks.

In some embodiments, the human patient survives with a lower rate of progression of the non-small cell lung cancer for at least 14 weeks.

In some embodiments, the human patient survives free of progression of the non-small cell lung cancer for at least 8 weeks.

In some embodiments, the human patient survives free of progression of the non-small cell lung cancer for at least 14 weeks.

In some embodiments, the human patient survives free of progression of the non-small cell lung cancer for at least 20 weeks.

In some embodiments, the human patient survives free of progression of the non-small cell lung cancer for at least 26 weeks.

In some embodiments, the human patient suffers from chest pain, pleural effusions, pulmonary edema, dyspnea, or hemoptysis.

In some embodiments, the non-small cell lung cancer is lung adenocarcinoma or lung large cell carcinoma.

In some embodiments, the non-small cell lung cancer of non-squamous histology is lung adenocarcinoma or lung large cell carcinoma.

In some embodiments, during the chemotherapy the amount of paclitaxel administered is 200 mg/m² intravenously to the human patient over a period of 3 hours.

In some embodiments, during the chemotherapy the amount of paclitaxel administered is less than 200 mg/m² intravenously to the human patient.

In some embodiments, during the chemotherapy the paclitaxel is administered to the human patient on the first day of each of up to six three-week chemotherapy cycles.

In some embodiments, during the chemotherapy the amount of carboplatin administered is AUC 6 mg/mL/min intravenously to the human patient over a period of 30 minutes.

In some embodiments, during the chemotherapy the amount of carboplatin administered is less than AUC 6 mg/mL/min intravenously to the human patient over a period of 30 minutes.

In some embodiments, during the chemotherapy the carboplatin is administered to the human patient on the first day of each of up to six three-week chemotherapy cycles.

In some embodiments, the anti-clusterin oligonucleotide is administered to the human patient intravenously in an aqueous solution comprising sodium ions.

In some embodiments, the anti-clusterin oligonucleotide is administered to the human patient 3 times within a 5 to 9 day period before the first day of chemotherapy and then once weekly beginning on the first day of chemotherapy.

In some embodiments, the human patient has not received treatment for non-small cell lung cancer for at least 1 year.

In some embodiments, the human patient has not received a chemotherapeutic agent for the treatment of non-small cell lung cancer for at least 1 year.

In some embodiments, the human patient is afflicted with non-small cell lung cancer of non-squamous histology.

In some embodiments, the human patient is afflicted with Stage IV non-small cell lung cancer.

In some embodiments, the human patient is afflicted with Stage IV non-small cell lung cancer of non-squamous histology.

In some embodiments, a method of the invention for treating a human patient afflicted with non-small cell lung cancer of non-squamous histology or Stage IV non-small cell lung cancer further comprises the steps of:
i) measuring the level of serum clusterin present in the blood of the human patient prior to the administration of the anti-clusterin oligonucleotide;
ii) determining whether the level of serum clusterin present in the human patient is lower than a predetermined upper threshold level of baseline serum clusterin below which a human patient is likely to substantially benefit from anti-clusterin therapy; and
iii) administering the anti-clusterin oligonucleotide only if the level of serum clusterin present in the blood of the human patient is lower than the predetermined upper threshold level of baseline serum clusterin.

In some embodiments, in step i) the measuring is performed after initiation of the chemotherapy.

In some embodiments, the predetermined upper threshold level of baseline serum clusterin is 75 μg/mL.

In some embodiments, a method of the invention for treating a human patient afflicted with non-small cell lung cancer of non-squamous histology or Stage IV non-small cell lung cancer further comprises the steps of:
i) administering to the human patient the anti-clusterin oligonucleotide in an initial dosage and treatment protocol;
ii) thereafter testing the human patient to determine a level of serum clusterin after a period of treatment with the anti-clusterin oligonucleotide intended to reduce clusterin expression;
iii) determining an adjusted dosage and treatment protocol based on the determined level of serum clusterin; and
iv) administering to the human patient the anti-clusterin) oligonucleotide in accordance with the adjusted dosage and treatment protocol.

In some embodiments, the determined level of serum clusterin after a period of treatment with the anti-clusterin oligonucleotide intended to reduce clusterin expression is above a predetermined post anti-clusterin oligonucleotide initiation threshold level.

In some embodiments, the predetermined post anti-clusterin oligonucleotide initiation threshold level is 30 μg/mL.

In some embodiments, the adjusted dosage and treatment protocol comprises administration of the anti-clusterin oligonucleotide to the human patient two or three times per week.

Some embodiments of the present invention provide a combination for treating a human patient afflicted with non-small cell lung cancer of non-squamous histology or Stage IV non-small cell lung cancer, comprising chemotherapy consisting of paclitaxel and carboplatin, and an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19.

Some embodiments of the present invention provide a composition for treating a human patient afflicted with non-small cell lung cancer of non-squamous histology or Stage IV non-small cell lung cancer, comprising chemotherapy consisting of paclitaxel and carboplatin, and an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19.

Some embodiments of the present invention provide a pharmaceutical composition for treating a human patient afflicted with non-small cell lung cancer of non-squamous histology or Stage IV non-small cell lung cancer, the composition comprising chemotherapy consisting of paclitaxel and carboplatin, and an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19.

Some embodiments of the present invention relate to the use of a composition comprising chemotherapy consisting of paclitaxel and carboplatin, and an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, for treatment of a human patient afflicted with non-small cell lung cancer of non-squamous histology or Stage IV non-small cell lung cancer.

Some embodiments of the present invention relate to the use of a composition comprising chemotherapy consisting of paclitaxel and carboplatin, and an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, for preparation of a medicament for treatment of a human patient afflicted with non-small cell lung cancer of non-squamous histology or Stage IV non-small cell lung cancer.

Some embodiments of the present invention provide a package for use in the treatment of a human patient afflicted with non-small cell lung cancer of non-squamous histology or Stage IV non-small cell lung cancer, comprising chemotherapy consisting of paclitaxel and carboplatin, and an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, and instructions for the use of the chemotherapy in combination with the anti-clusterin oligonucleotide for the treatment of non-small cell lung cancer of non-squamous histology or Stage IV non-small cell lung cancer.

Some embodiments of the present invention provide a chemotherapy consisting of paclitaxel and carboplatin, for use in combination with an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, for treating of a human patient afflicted with non-small cell lung cancer of non-squamous histology or Stage IV non-small cell lung cancer; or an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, for use in combination with a chemotherapy consisting of paclitaxel and carboplatin, for treating of a human patient afflicted with non-small cell lung cancer of non-squamous histology or Stage IV non-small cell lung cancer.

In some embodiments, treatment encompasses the human patient being free of progression of NSCLC of non-squamous histology. In some embodiments, treatment encompasses the human patient being substantially free of progression of NSCLC of non-squamous histology. In some embodiments, the human patient is free of progression of measurable disease. In some embodiments, the human patient is free of progression of non-measurable disease. In some embodiments, treatment of the human patient encompasses the prevention or amelioration of a symptom of NSCLC of non-squamous histology.

In some embodiments, treatment encompasses the human patient being free of progression of Stage IV NSCLC. In some embodiments, treatment encompasses the human patient being substantially free of progression of Stage IV NSCLC. In some embodiments, treatment of the human patient encompasses the prevention or amelioration of a symptom of Stage IV NSCLC.

In some embodiments, the time to progression of NSCLC is increased.

In some embodiments, the cells of the lung cancer comprise an epidermal growth factor (EGFR) mutation. In some embodiments, the cells of the lung cancer comprise a v-Ki-ras2 Kirsten rat sarcoma viral ongocene homolog (KRAS) mutation.

In some embodiments, the human patient has histologically or cytologically confirmed, unresectable advanced or metastatic NSCLC.

In some embodiments, the human patient has a life expectancy of at least 12 weeks from the initiation of treatment.

In some embodiments, the human patient has received at least one prior line of platinum-based systemic anticancer therapy for advanced or metastatic NSCLC.

In some embodiments, the human patient has documented radiological disease progression during first-line therapy.

In some embodiments, the human patient has documented radiological disease progression after first-line therapy.

In some embodiments, the human patient has adequate electrolyte values, bone marrow, renal and liver functions within 1, 2, 3, 4, 5, 6, 7, 8, 9, 10, 11 or 12 weeks of treatment initiation as defined below:
Absolute neutrophil count (ANC)≧1.5×109/L
Platelets≧100×109/L
Hemoglobin≧9 g/dL
Serum creatinine≦1.5× upper limit of normal (ULN)
Total Bilirubin≦1.0×ULN (unless elevated secondary to benign conditions such as Gilbert's disease)
AST and ALT≦1.5×ULN
Alkaline phosphatase≦2.5 ULN
Electrolyte values (sodium, potassium and magnesium)≧1×LLN and ≦1×ULN. Patients with corrected electrolyte values are eligible.

The present invention also provides a method of treating a human patient afflicted with unresectable, advanced or metastatic non-small cell lung cancer comprising periodically administering to the human patient chemotherapy comprising an amount of docetaxel; and 640 mg of an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, thereby treating the human patient afflicted with unresectable, advanced or metastatic non-small cell lung cancer.

In some embodiments, the treating includes prolonging survival of the human patient.

In some embodiments, the treating includes prolonging survival of the human patient which prolonged survival is free of progression of the non-small cell lung cancer.

In some embodiments, the human patient survives free of progression of the non-small cell lung cancer for at least 14 weeks.

In some embodiments, the human patient survives free of progression of the non-small cell lung cancer of non-squamous histology for at least 14 weeks.

In some embodiments, the human patient suffers from chest pain, pleural effusions, pulmonary edema, dyspnea, or hemoptysis.

In some embodiments, the non-small cell lung cancer is lung adenocarcinoma or lung large cell carcinoma.

In some embodiments, the non-small cell lung cancer of non-squamous histology is lung adenocarcinoma or lung large cell carcinoma.

In some embodiments, during the chemotherapy the amount of docetaxel administered is 75 mg/m² intravenously to the human patient over a period of 1 hour.

In some embodiments, during the chemotherapy the amount of docetaxel administered is less than 75 mg/m² intravenously to the human patient.

In some embodiments, during the chemotherapy the docetaxel is administered to the human patient on the first day of each of at least one three-week chemotherapy cycle.

In some embodiments, the anti-clusterin oligonucleotide is administered to the human patient intravenously in an aqueous solution comprising sodium ions.

In some embodiments, the anti-clusterin oligonucleotide is administered to the human patient 3 times within a 5 to 9 day period before the first day of chemotherapy and then once weekly beginning on the first day of chemotherapy.

In some embodiments, the lung cancer is nonresectable, advanced or metastatic non-small cell lung cancer.

In some embodiments, the lung cancer has been histologically or cytologically confirmed and is, unresectable, advanced or metastatic (Stage IV per AJCC 7^{th} edition TNM staging).

In some embodiments, the lung cancer is Stage IV disease (according to the IASLC 7^{th} edition TNM staging, including subjects with pleural effusion who were previously classified as Stage IIIB) that is not amenable to either surgery or radiation therapy of curative intent.

In some embodiments, the human patient has not received treatment for non-small cell lung cancer for at least 1 year.

In some embodiments, the human patient has not received a chemotherapeutic agent for the treatment of non-small cell lung cancer for at least 1 year.

In some embodiments, the human patient has before initiation of the periodic administration received a chemotherapeutic agent for the treatment of lung cancer.

In some embodiments, the chemotherapeutic agent was a platinum-based chemotherapeutic agent.

In some embodiments, the human patient is afflicted with non-small cell lung cancer of non-squamous histology.

In some embodiments, the human patient is afflicted with Stage IV non-small cell lung cancer of non-squamous histology.

In some embodiments, a method of the invention for treating a human patient afflicted with non-small cell lung cancer of non-squamous histology or Stage IV non-small cell lung cancer further comprises the steps of:
i) measuring the level of serum clusterin present in the blood of the human patient prior to the administration of the anti-clusterin oligonucleotide;
ii) determining whether the level of serum clusterin present in the human patient is lower than a predetermined upper threshold level of baseline serum clusterin below which a human patient is likely to substantially benefit from anti-clusterin therapy; and
iii) administering the anti-clusterin oligonucleotide only if the level of serum clusterin present in the blood of the human patient is lower than the predetermined upper threshold level of baseline serum clusterin.

In some embodiments, in step i) the measuring is performed after initiation of the chemotherapy.

In some embodiments, the predetermined upper threshold level of baseline serum clusterin is 75 μg/mL.

In some embodiments, a method of the invention for treating a human patient afflicted with non-small cell lung cancer of non-squamous histology or Stage IV non-small cell lung cancer further comprises the steps of:
i) administering to the human patient the anti-clusterin oligonucleotide in an initial dosage and treatment protocol;
ii) thereafter testing the human patient to determine a level of serum clusterin after a period of treatment with the anti-clusterin oligonucleotide intended to reduce clusterin expression;
iii) determining an adjusted dosage and treatment protocol based on the determined level of serum clusterin; and
iv) administering to the human patient the anti-clusterin oligonucleotide in accordance with the adjusted dosage and treatment protocol.

In some embodiments, the determined level of serum clusterin after a period of treatment with the anti-clusterin oligonucleotide intended to reduce clusterin expression is above a predetermined post anti-clusterin oligonucleotide initiation threshold level.

In some embodiments, the predetermined post anti-clusterin oligonucleotide initiation threshold level is 30 μg/mL.

In some embodiments, the adjusted dosage and treatment protocol comprises administration of the anti-clusterin oligonucleotide to the human patient two or three times per week.

The present invention also provides a combination for treating a human patient afflicted with unresectable, advanced or metastatic non-small cell lung cancer, comprising chemotherapy comprising docetaxel; and an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19. In some embodiments, the combination is for treating a human patient afflicted with non-small cell lung cancer of non-squamous histology or Stage IV non-small cell lung cancer.

The present invention also provides a composition for treating a human patient afflicted with unresectable, advanced or metastatic non-small cell lung cancer, comprising chemotherapy comprising docetaxel; and an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19. In some embodiments, the composition is for treating a human patient afflicted with non-small cell lung cancer of non-squamous histology or Stage IV non-small cell lung cancer.

The present invention also provides a pharmaceutical composition for treating a human patient afflicted with unresectable, advanced or metastatic non-small cell lung cancer, the composition comprising chemotherapy comprising docetaxel; and an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19. In some embodiments, the pharmaceutical composition is for treating a human patient afflicted with non-small cell lung cancer of non-squamous histology or Stage IV non-small cell lung cancer.

Some embodiments of the present invention relate to the use of a composition comprising chemotherapy comprising docetaxel; and an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, for treatment of a human patient afflicted with unresectable, advanced or metastatic non-small cell lung cancer. In some embodiments, the use of the composition is for treatment of a human patient afflicted with non-small cell lung cancer of non-squamous histology or Stage IV non-small cell lung cancer.

Some embodiments of the present invention relate to the use of a composition comprising chemotherapy comprising docetaxel; and an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, for preparation of a medicament for treatment of a human patient afflicted with unresectable, advanced or metastatic non-small cell lung cancer. In some embodiments, the use of the composition is for preparation of a medicament for treatment of a human patient afflicted with non-small cell lung cancer of non-squamous histology or Stage IV non-small cell lung cancer.

The present invention also provides a package for use in the treatment of a human patient afflicted with unresectable, advanced or metastatic non-small cell lung cancer, comprising chemotherapy comprising docetaxel; and an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, and instructions for the use of the chemotherapy in combination with the anti-clusterin oligonucleotide for the treatment of unresectable, advanced or metastatic non-small cell lung cancer. In some embodiments, the package is for use in the treatment of a human patient afflicted with non-small cell lung cancer of non-squamous histology or Stage IV non-small cell lung cancer.

The present invention also provides a chemotherapy comprising docetaxel for use in combination with an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, for treating of a human patient afflicted with unresectable, advanced or metastatic non-small cell lung cancer; or an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, for use in combination with a chemotherapy comprising docetaxel, for treating of a human patient afflicted with unresectable, advanced or metastatic non-small cell lung cancer. In some embodiments, the chemotherapy in combination with the anti-clusterin oligonucleotide is for treating a human patient afflicted with non-small cell lung cancer of non-squamous histology or Stage IV non-small cell lung cancer. In some embodiments, the anti-clusterin oligonucleotide in combination with the chemotherapy is for treating a human patient afflicted with non-small cell lung cancer of non-squamous histology or Stage IV non-small cell lung cancer.

It is understood that where a parameter range is provided, all integers within that range, and tenths thereof, are also provided by the invention. For example, “0.2-5 mg/kg/day” is a disclosure of 0.2 mg/kg/day, 0.3 mg/kg/day, 0.4 mg/kg/day, 0.5 mg/kg/day, 0.6 mg/kg/day etc. up to 5.0 mg/kg/day.

### Taxanes

Taxanes are a class of chemotherapeutic including paclitaxel, docetaxel, baccatin III, baccatin V, taxol B (cephalomannine), taxol C, taxol D, taxol E, taxol F, taxol G, cabazitaxel, larotaxel, ortataxel (14 beta-hydroxydeacetyl baccatin III), tesetaxol, 10-deacetyl baccatin III, 7-xylosyl-10-deacetyl cephalomannine, 7-xylosyl-10-deacetyl paclitaxel, 10-deacetyl cephalomannine, 7-xylosyl-10-deacetyl taxol C, 10-deacetyl paclitaxel, 7-xylosyl paclitaxel, 10-deacetyl taxol C, 10-deacetyl-7-epi cephalomaunine, 7-xylosyl taxol C, 10-deacetyl-7-epipaclitaxel, 7-epi cephalomaunine, 7-epi paclitaxel, 7-O-methylthiomethyl paclitaxel, 7-deoxy docetaxel, taxanime M, PG-paclitaxel, DHA-paclitaxel.

Taxanes have been approved by the FDA including paclitaxel (e.g., for NSCLC, AIDS-related Kaposi sarcoma, breast cancer and ovarian cancer), cabazitaxel (e.g., for prostate cancer), and docetaxel (e.g., for NSCLC, breast cancer, gastric (stomach) cancer, prostate cancer, squamous cell carcinoma of the head and neck).

Taxanes also include derivatives of these compounds, particularly ester and ether derivatives and pharmaceutically acceptable salts thereof. Taxanes may also include any drug or derivative of a drug which has a carbon framework substantially identical to the framework of the above taxanes.

Without being bound to any particular theory, taxanes may achieve their therapeutic effect by interfering with cell division by stabilizing tubulin in the microtubule. Taxanes may be naturally occurring, semi-synthetic, or synthetic compounds. Semi-synthetic taxanes may be prepared by modification of a known or naturally occurring taxane. The taxanes may be prepared as a fatty acid-bound, peptide-bound, albumin-bound or other protein-bound suspension or dissolved in a solution, such as polyoxyl 35 or polysorbate 80.

### Paclitaxel

Paclitaxel is sold under the brand names Taxol® and Abraxane®, and has been used for the treatment of NSCLC (Taxol® Package Insert, Bristol-Myersw Squibb Company (Princeton, N.J., USA); D'Addario et al., 2010; National Comprehensive Cancer Network Clinical Practice Guidelines in Oncology, Non-Small Cell Lung Cancer, V.2.2010).

Paclitaxel is known to cause several side effects. Neutropenia, the most frequent side effect, is profound but generally of short duration. Peripheral neuropathy, myalgia, and arthralgia are usually noted with the administration of higher doses of paclitaxel 175 mg/m²) for several cycles. Paclitaxel can cause rapid and complete alopecia. Other toxicities include: mild to moderate nausea, vomiting, diarrhea, and mucositis.

For paclitaxel therapy, standard steroid premedication to prevent severe hypersensitivity reactions and antiemetics may be given according to institutional practice. According to the package insert, the recommended premedication consists of dexamathasone 20 mg p.o. administered twice, approximately 12 and 6 hours before paclitaxel, diphenhydramine (or its equivalent) 50 mg i.v./.p.o. 30 to 60 minutes prior to paclitaxel, and cimetidine (300 mg) or ranitidine (50 mg) i.v./p.o. 30 to 60 minutes prior to paclitaxel.

### Docetaxel

Docetaxel is sold under the brand name Taxotere® and has been used for second-line treatment of NSCLC (Taxotere® Prescribing Information, Sanofi-Aventis LLC, May 2010, (Bridgewater, N.J., USA).

Docetaxel has also been used as treatment for metastatic breast cancer, early-stage breast cancer and metastatic androgen independent prostate cancer.

Docetaxel is known to cause several side effects, the most common of which are infections, neutropenia, anemia, febrile neutropenia, hypersensitivity, thrombocytopenia, neuropathy, dysgeusia, dyspnea, constipation, anorexia, nail disorders, fluid retention, asthenia, pain, nausea, diarrhea, vomiting, mucositis, alopecia, skin reactions and myalgia.

Neutropenia (<2,000 neutrophils/mm³) occurs in virtually all patients given 60-100 mg/m² of Docetaxel and grade 4 neutropenia (<500 cells/mm³) occurs in 85% of patients given 100 mg/m² and 75% of patients given 60 mg/m².

The incidence of treatment-related mortality associated with Docetaxel therapy is increased in patients with abnormal liver function, in patients receiving higher doses, and in patients with non-small cell lung carcinoma and a history of prior treatment with platinum-based chemotherapy who receive Taxotere® as a single agent at a dose of 100 mg/m².

Patients may be premedicated with corticosteroids, such as dexamethasone, to each Docetaxel administration to reduce the incidence of and severity of fluid retention.

Docetaxel may be prescribed as a one-hour infusion every three weeks or as weekly administration (John D. Hainsworth, “Practical Aspects of Weekly Docetaxel Administration Schedules” September 2004, vol. 9, no. 5, 538-545)

### Platinum-Based Chemotherapeutic Agents

Platinum-based chemotherapeutic agents are a class of chemotherapy drugs. Platinum-based chemotherapeutic agents include cisplatin, carboplatin (also known as paraplatin), nedaplatin, oxaliplatin, triplatin tetranitrate, satraplatin, iproplatin, lobaplatin, picoplatin and combinations thereof. Platinum-based chemotherapeutic agents are approved by the FDA and include cisplatin (NSCLC, bladder cancer, cervical cancer, malignant mesothelioma, ovarian cancer, squamous cell carcinoma of the head and neck, and testicular cancer), oxaliplatin (colorectal cancer and stage III colon cancer), and carboplatin (NSCLC and ovarian cancer) are approved by the FDA.

Platinum-based chemotherapeutic agents also include derivatives of these compounds, particularly ester and ether derivatives and pharmaceutically acceptable salts thereof. Platinum-based chemotherapeutic agents may also include any drug or derivative of a drug which has a carbon framework substantially identical to the framework of the above platinum-based chemotherapeutic agents.

Without being bound to any particular theory, platinum-based chemotherapeutic agents can be classified as alkylating or alkylating-like agents because they interact with DNA irreversibly through cross-linking and platinum-DNA adduct forming reactions which prevent DNA repair or replication and result in apoptosis of cells.

Common side-effects of platinum-based chemotherapeutic agents include nephrotoxicity, neurotoxicity, nausea and vomiting, ototoxicity, electrolyte disturbance, myelotoxicity, and hemolytic anemia.

### Carboplatin

Carboplatin is sold under the brand name Paraplatin®, and has been used for the treatment of NSCLC (Carboplatin Package Insert, Bedford Labs (Bedford, Ohio, USA); D'Addario et al., 2010; National Comprehensive Cancer Network Clinical Practice Guidelines in Oncology, Non-Small Cell Lung Cancer, V.2.2010).

Bone marrow suppression is the major dose-limiting toxicity of carboplatin. Nausea, vomiting, and loss of appetite are usually mild to moderate. Less common adverse events includes ototoxicity, nephrotoxicity, neurotoxicity, hypomagnesemia, edema, alopecia, amenorrhea, CNS toxicity (dizziness, blurred vision), hypercalcemia, abnormal liver function tests, allergic reactions, and veno-occlusive disease. For full safety information, please refer to the carboplatin package insert, a copy of which is incorporated herein by reference.

### TERMS

As used herein, and unless stated otherwise, each of the following terms shall have the definition set forth below.

As used herein, “anti-clusterin therapy” is therapy which reduces the expression of clusterin. An anti-clusterin therapy may be an anti-clusterin oligonucleotide.

Antisense oligonucleotides (ASOs) are stretches of single-strand deoxyribonucleic acid (DNA) complementary to messenger ribonucleic acid (mRNA) regions of a target gene. Because cellular ribosomal machinery translates mRNA into proteins, expression of specific proteins can be reduced by blocking or reducing this translation.

As used herein, “anti-clusterin oligonucleotide” refers to an antisense oligonucleotide which reduces clusterin expression, and comprises a nucleotide sequence that is complementary to clusterin-encoding mRNA. An example of an anti-clusterin oligonucleotide is custirsen.

As used herein, “custirsen” refers to an anti-clusterin oligonucleotide having nucleotides in the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19. Custirsen can be in the form of Custirsen Sodium.

As used herein, “a human patient afflicted with” a condition, e.g. non-small cell lung cancer, means a human patient who was been affirmatively diagnosed to have the condition.

As used herein, a cancer with “non-squamous histology” is a cancer that is not predominantly of squamous histology as determined by histological methods known in the art. Subtypes of NSCLC of non-squamous histology include but are not limited to lung adenocarcinoma, and lung large cell carcinoma. As used herein, “squamous” means derived from, originating from, and/or consisting of a stratified epithelium that predominantly comprises squamous cells.

As used herein, “predominantly of squamous histology” means >50% of squamous histology as determined by histological methods known in the art.

As used herein, a cancer with “squamous histology” is a cancer with >50% squamous histology as determined by histological methods known in the art. A non-limiting example of a lung cancer which has squamous histology is squamous cell lung cancer, which is a type of non-small cell lung cancer.

Aspects of the invention may be applied to the treatment of NSCLC that has metastasized, or is metastasizing through various routes, including, but not limited to the lymph nodes.

As used herein, “taxane/platinum-based chemotherapeutic agent” means a taxane and a platinum-based chemotherapeutic agent.

As used herein, “paclitaxel/carboplatin” means paclitaxel and carboplatin.

As used herein, “docetaxel/platinum-based chemotherapy” means docetaxel and a platinum-based chemotherapeutic agent.

“Combination” means either at the same time and frequency, or more usually, at different times and frequencies as custirsen, as part of a single treatment plan. Aspects of the invention include the administration of custirsen before, after, and/or during the administration of the taxane and/or a platinum-based chemotherapeutic agent. Furthermore, aspects of the invention include the administration of custirsen before, after, and/or during the administration of carboplatin. A taxane and a platinum-based chemotherapeutic agent may therefore be used, in combination with custirsen according to the invention, but yet be administered at different times, different dosages, and at a different frequency, than custirsen and/or each other. Aspects of the invention also include the administration of custirsen before, after, and/or during the administration of a taxane and/or a platinum-based chemotherapeutic agent. A taxane and/or a platinum-based chemotherapeutic agent may therefore be used, in combination with custirsen according to the invention, but yet be administered at different times, different dosages, and at a different frequency, than custirsen and/or each other. For example, paclitaxel and carboplatin may be used, in combination with custirsen according to the invention, but yet be administered at different times, different dosages, and at a different frequency, than custirsen and/or each other. As another example, docetaxel may be used, in combination with custirsen according to the invention, but yet be administered at different times, different dosages, and at a different frequency, than custirsen and/or each other.

As used herein, “lung adenocarcinoma” encompasses any malignant epithelial NSCLC which has glandular and/or duct differentiation, and excludes any NSCLC that is not predominantly non-squamous. Non-limiting examples of subdivisions of the lung adenocarcinoma subtype of NSCLC are acinar, papillary, BAC, and solid adenocarcinoma with mucin production. One of skill in the art will recognize that lung adenocarcinomas comprising combinations of two or more of these or other subdivisions are common.

As used herein, “lung large cell carcinoma” means a NSCLC of non-squamous histology that is not lung adenocarcinoma.

One of skill in the art will realize that NSCLC, as well as its subtypes, including lung adenocarcinoma and lung large cell carcinoma, are heterogeneous with multiple histological variants. Therefore, the term “non-small cell lung cancer of non-squamous histology” encompasses all types and subdivisions of NSCLC that are predominantly non-squamous.

As used herein, “Stage IV non-small cell lung cancer” means NSCLC comprising a tumor, wherein i) the NSCLC has metastasized to another region of the body outside the lungs or to a contralateral lobe of the lungs, and/or ii) there is malignant pleural effusion, malignant pericardial effusion, and/or a pleural nodule.

As used herein, “lesion” means a NSCLC growth or tumor.

The finding of a “new lesion” should be unequivocal, i.e. not attributable to difference in scanning technique, change in imaging modality, or findings thought to represent something other than cancer growth (e.g. some new bone lesions may be simply healing or a flare of pre-existing lesions; or necrosis of a liver lesion may be reported on a CT scan report as a “new” cystic lesion without being a “new lesion” as used herein).

As used herein, “measurable disease” means having a NSCLC tumor with at least one dimension (longest diameter to be recorded) of at least 10 mm by CT scan, MRI or caliper measurement, or a malignant lymph node ≧15 mm in short axis by CT scan, MRI or caliper measurement.

All other NSCLC lesions, including small tumors (longest diameter<10 mm or pathological lymph nodes with >10 to <15 mm short axis) are considered “non-measurable disease” as used herein. Lesions considered truly non-measurable include: leptomeningeal disease, malignant ascites, malignant pleural or pericardial effusion, inflammatory breast disease, lymphangitic involvement of skin or lung, abdominal masses/abdominal organomegaly identified by physical exam that is not measurable by reproducible imaging techniques. Bone-lesions: Bone scan, PET scan or plain films are not considered adequate imaging techniques to measure bone lesions. However, these techniques can be used to confirm the presence or disappearance of bone lesions.

As used herein, “progression of measurable disease” will have occurred if there is an increase of at least 20% in the sum of the longest diameter(s) of all measurable lesion(s), taking as reference the smallest sum recorded since the beginning of treatment (baseline or nadir), wherein the sum has an absolute increase of at least 5 mm, or there is an appearance of one or more new soft tissue (visceral or nodal) measurable lesions after treatment has begun. These criteria should be met on chest, abdomen, or pelvic CT scan(s) or MRI, unless otherwise specified, such as by caliper measurement.

As used herein, “progression of non-measurable disease” will have occurred if there is an appearance of one or more new lesions that does not qualify as progression of measurable disease.

As used herein, “metastasis involving lymph nodes” means having a malignant lymph node that was not previously irradiated and is >15 mm in short axis when assessed by CT scan, MRI, or caliper measurement.

As used herein, “time to progression” of measurable disease is the amount of time between the beginning of treatment and the progression of measurable disease. “Time to progression” of non-measurable disease is the amount of time between the beginning of treatment and the progression non-measurable disease.

As used herein, “free of progression of measurable disease” means that there has not been is an increase of at least 20% in the sum of the longest diameter(s) of all measurable lesion(s), taking as reference the smallest sum recorded since the beginning of treatment (baseline or nadir), wherein the sum has an absolute increase of at least 5 mm, and there has not been an appearance of one or more new soft tissue (visceral or nodal) tumor lesions after treatment has begun, as determined by chest, abdomen, or pelvic CT scan(s) or MRI, unless otherwise specified, such as by caliper measurement.

As used herein, “free of progression of non-measurable disease” means that there has been no appearance of one or more new lesions that are considered non-measurable.

As used herein, “free of progression of the non-small cell lung cancer” means free of progression of both measurable and non-measurable disease.

As used herein, “rate of progression of the non-small cell lung cancer” means the frequency at which progression of measurable disease and/or non-measurable disease is observed over the course of two or more time points following an initial, baseline observation. Progression of measurable disease and/or non-measurable disease may be determined at various time points, including weekly, monthly, and/or at any other time point and/or points indicated. Non-limiting examples of time points at which measurable and/or non-measurable disease may be determined include any week which is 1-26 weeks after the initiation of treatment with custirsen and a taxane, or custirsen and a taxane and a platinum-based chemotherapeutic agent, or custirsen and/or paclitaxel/carboplatin, or custirsen and docetaxel, or custirsen and docetaxel, such as at 8, 14, 20, and/or 26 weeks.

As used herein, “substantial progression of measurable disease” will have occurred if there is an increase of at least 30% in the sum of the longest diameter(s) of all measurable lesion(s), taking as reference the smallest sum recorded since the beginning of treatment (baseline or nadir), wherein the sum has an absolute increase of at least 7.5 mm after treatment has begun. These criteria should be met on chest, abdomen, or pelvic CT scan(s) or MRI, unless otherwise specified.

As used herein, an “amount” or “dose” of custirsen as measured in milligrams refers to the milligrams of custirsen present in a preparation, regardless of the form of the preparation.

As used herein, “effective” when referring to an amount of a taxane, a platinum-based chemotherapeutic agent, custirsen, paclitaxel, docetaxel, or carboplatin, or any combination thereof refers to the quantity of taxane, a platinum-based chemotherapeutic agent, custirsen, paclitaxel, docetaxel, or carboplatin, or any combination thereof that is sufficient to yield a desired therapeutic response without undue adverse side effects (such as toxicity, irritation, or allergic response) commensurate with a reasonable benefit/risk ratio when used in the manner of this invention.

As used herein, “treating” encompasses, e.g., inhibition, regression, or stasis of the progression of NSCLC. Treating also encompasses the prevention or amelioration of any symptom or symptoms of NSCLC.

As used herein, “inhibition” of disease progression or disease complication in a subject means preventing or reducing the disease progression and/or disease complication or symptom in the subject.

As used herein, a “symptom” associated with NSCLC includes any clinical or laboratory manifestation associated with NSCLC and is not limited to what the subject can feel or observe. Symptoms of NSCLC include but are not limited to chest pain, pleural effusions, pulmonary edema, dyspnea, hemoptysis, wheezing, cachexia, shortness of breath, and dysphagia.

As used herein, an “adverse event” or “AE” means any untoward medical occurrence in a clinical trial subject administered a medicinal product and which does not have a causal relationship with the treatment. An adverse event can therefore be any unfavorable and unintended sign including an abnormal laboratory finding, symptom, or diseases temporally associated with the use of an investigational medicinal product, whether or not considered related to the investigational medicinal product. A new condition or the worsening of a pre-existing condition may be considered an AE. Stable chronic conditions such as arthritis that is present prior to study entry and do not worsen during treatment are not considered AEs. Worsening of the disease may be measured by clinical and radiological parameters, and is only an AE if the outcome is more serious than would normally be expected from the normal course of the disease in a particular subject.

As used herein, “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject. An example of a pharmaceutically acceptable carrier is a nanoparticle. The nanoparticle may be a protein, such as albumin. A taxane, such as Docetaxel or Paclitaxel, and/or a platinum-based chemotherapeutic agent, such as carboplatin, may be conjugated to a nanoparticle. An example of a taxane bound to a nanoparticle includes, but is not limited to nanoparticle paclitaxel (nab-paclitaxel), which is sold under the brand name Abraxane®.

The following abbreviations are used herein:
AE Adverse Event
ALT Alanine Transaminase (SGPT)
ANC Absolute Neutrophil Count
ASCO American Society of Clinical Oncology
ASO Antisense Oligonucleotide
AST Aspartate Transaminase (SGOT)
AUC Area Under the Curve
AV Atrioventricular
AWP Alive Without Progression
β hCG Beta Human chorionic gonadotropin
BSA Body Surface Area
CA Competent Authority
CDMS Clinical Data Management System
CRA Clinical Research Associate
CRF Case Report Form
CRO Clinical Research Organization
CRPC Castrate Resistant Prostate Cancer
CSU Clinical Supplies Unit
CT Computed Tomography
CTCAE Common Terminology Criteria for Adverse Events
CVA Cerebrovascular Accident
dl Deciliter
DNA Deoxyribonucleic Acid
DMC Data Monitoring Committee
EC Ethics Committee
ECG Electrocardiogram
ECOG Eastern Cooperative Oncology Group
EDC Electronic Data Capture
EGFR Epidermal Growth Factor Receptor
EMA European Medicines Agency
EOI End of Infusion
ERCC-1 Excision Repair Cross Complementation Group 1
EU European Union
FDA Food and Drug Administration
GCP Good Clinical Practice
G-CSF Granulocyte Colony Stimulating Factor
GFR Glomerular Filtration Rate
GGT Gamma Glutamyltransferase
HR Heart Rate/Hazard Ratio
IASLC International Association for the Study of Lung Cancer
IB Investigator's Brochure
ICF Informed Consent Form
ICH International Conference on Harmonization
IND Investigational New Drug
IMP Investigational Medicinal Product
IRB Institutional Review Board
IR&D Innovative Research and Development
ITT Intent to Treat
IV Intravenous
IVRS Interactive Voice Response System
IWRS Interactive Web Response System
K Potassium
kg Kilogram
LCM Local Clinical Management
LD Longest Diameter
LDH Lactate Dehydrogenase
LFT Liver Function Tests
m² Meter Squared
MedRA Medical Dictionary for Regulatory Activities
mg Milligram
min Minute
ml Milliliter
MOE Methoxyethyl
MRI Magnetic Resonance Imaging
Na Sodium
NCl National Cancer Institute
NSAIDs Nonsteroidal Anti-inflammatory Drugs
NSCLC Non-small Cell Lung Cancer
OS Overall Survival
PE Physical Examination
PET Positron Emission Tomography
PO Per Os
PFS Progression Free Survival
QA Quality Assurance
RBC Red Blood Cell (Count)
RECIST Response Evaluation Criteria In Solid Tumors
RNA Ribonucleic Acid
SAE Serious Adverse Event
SAP Statistical Analysis Plan
SD Standard Deviation
SGOT Serum Glutamate Oxaloacetate Transaminase
SGPT Serum Glutamate Pyruvate Transaminase
SOI Start of Infusion
SOP Standard Operating Procedure
SUSAR Suspected Unexpected Serious Adverse Reaction
TNM TNM Classification of Malignant Tumors (Tumor, Nodes, Metastases)
ULN Upper Limit of Normal
WBC White Blood Cell (Count)
WHO World Health Organization

In some embodiments of the invention, the anti-tumor activity of the taxane regimen is enhanced when combined with custirsen-induced clusterin suppression. In some embodiments of the invention, the anti-tumor activity of the taxane/platinum-based chemotherapeutic agent regimen is enhanced when combined with custirsen-induced clusterin suppression. In some embodiments of the invention, the anti-tumor activity of the paclitaxel/carboplatin regimen is enhanced when combined with custirsen-induced clusterin suppression. In some embodiments of the invention, the anti-tumor activity of a docetaxel regimen is enhanced when combined with custirsen-induced clusterin suppression. Since suppressing clusterin expression may in turn lead to increased apoptosis, custirsen has effect on disease progression and survival in advanced NSCLC as described herein.

### Threshold Levels of Baseline Serum Clusterin

The methods of the present invention include performing at least one test to determine a level of serum clusterin. This test may be done to determine a “baseline level of serum clusterin” which is the level of clusterin present in the human patient prior to the initiation of treatment intended to reduce clusterin expression.

As used herein, an “upper threshold level” refers to a baseline level of serum clusterin present in a human patient below which the human patient is likely to substantially benefit from anti-clusterin therapy. In the present invention, there is a statistically significant difference in the efficacy of treatment of lung cancer (for example, NSCLC) between populations below and above the upper threshold level. To “substantially benefit from anti-clusterin therapy” means to exhibit treatment of a symptom of lung cancer, the degree of amelioration or prevention of which is improved when compared to a representative patient whose baseline clusterin levels are above the upper threshold level. For example, to substantially benefit from anti-clusterin therapy may mean having prolonged survival as compared to a representative patient whose baseline clusterin levels are above the upper threshold level.

In some embodiments of the invention, a decision on whether to treat the human patient with custirsen is made based on the whether the measured baseline value is above or below a predetermined threshold level of serum clusterin. In some embodiments, this threshold is between 30 and 75 μg/mL, although a person skilled in the art will recognize that the selection of specific threshold values may be dependent on the type of lung cancer, and also on the level of predictability of therapeutic efficacy that is desired.

In various embodiments of the present invention, a determination of baseline clusterin level is made and compared to a predetermined threshold. The threshold value is determined by a statistical analysis of data for a population of patients for whom both baseline clusterin levels, and periods of survival are known. It will be appreciated that the specific numerical value may be refined as more data becomes available. Furthermore, the specific numerical value employed will depend on the level of predictability of extended survival that is desired. Thus, if one wishes to be very sure that the use of custirsen will provide for longer survival, then a lower threshold value would be selected than if only a reasonable expectation of longer survival is required.

In some embodiments of the invention, the threshold value is selected as the median baseline clusterin value for a population of patients without selection for eventual survival time.

In some embodiments of the invention, the threshold value is determined by fitting baseline values and survival data a statistical model such as the Cox proportional hazards (PH) model with baseline clusterin as sole predictor.

In some embodiments of the invention, the threshold level of baseline serum clusterin is between 30 and 75 μg/mL. The threshold level of baseline serum clusterin may be 30, 35, 40, 45, 50, 55, 60, 65, 70, or 75 μg/mL, or any level between any of these possible levels.

In some embodiments of the invention, the threshold level of baseline serum clusterin is 30 μg/mL.

In some embodiments of the invention, the threshold level of baseline serum clusterin is 45 μg/mL.

In some embodiments of the invention, the threshold level of baseline serum clusterin is 55 μg/mL.

In some embodiments of the invention, the threshold level of baseline clusterin is 75 μg/mL.

In some aspects of the invention, the level of serum clusterin may be determined at a time after initiation of treatment with an anti-clusterin oligonucleotide such as custirsen. Testing the level of serum clusterin may be performed once or multiple times for a human patient. Suitably, this test is performed, one day, one week, two weeks, three weeks or one month after the initiation of treatment with custirsen, or multiple tests may be performed at weekly, biweekly, tri-weekly or monthly intervals. In some embodiments, tests are performed before the administration of chemotherapy, such as a taxane or a taxane and a platinum-based chemotherapeutic agent. In some embodiments, tests are performed after the administration of chemotherapy. In some embodiments, tests are performed at the beginning or end of one or more chemotherapy cycles comprising, e.g. taxane/platinum-based chemotherapeutic agent, or paclitaxel/carboplatin, or docetaxel during which the human patient also receives custirsen.

Based on the results of the serum clusterin determination, an effective dosage amount and schedule for custirsen is selected. When a post-treatment level of serum clusterin is determined and used, the effective dosage amount and schedule is referred to herein as an “adjusted dosage and treatment protocol.” The “adjusted dosage and treatment protocol” provides custirsen to the human patient at levels that are predicted to have optimized therapeutic efficacy in view of the serum clusterin levels.

Once a determination of serum clusterin is made after the initiation of treatment with custirsen, an effective dosage and schedule are determined that takes the serum clusterin value into account in order to maximize survival duration for the human patient. In general, higher levels of serum clusterin indicate a higher dosage and/or more frequent custirsen administration, provided the dosage of custirsen does not exceed 640 mg. The specific dosage and schedule that is selected will depend on a number of factors, including the human patient being treated. In some cases, a “post anti-clusterin oligonucleotide initiation threshold” value may be set which is indicative of a good prognosis for effective therapy. The post anti-clusterin oligonucleotide initiation threshold may also be referred to as a “post custirsen initiation threshold”. In this case, human patients below this threshold may be treated with a base custirsen dose/protocol. In some embodiments, a suitable base custirsen dose/protocol is 640 mg of custirsen per dose, independent of the weight of the human patient, with an administration schedule of once a week, optionally preceded by an initial loading of three doses in the first week. Human patients with post custirsen initiation serum clusterin levels higher than the post custirsen initiation threshold are suitably treated with more frequent dosages, for example twice or three times a week even after the loading week. In some embodiments of the invention, a dosage of custirsen lower than 640 mg custirsen is delivered more frequently than once per week following the loading week. This same post custirsen initiation threshold, or a different threshold value, may be used if the initiation of chemotherapy (e.g. with paclitaxel/carboplatin or docetaxel) is to be delayed during an initial period of clusterin reduction. Such a period may be one, two or three weeks, or until the baseline serum clusterin measurement drops below a determined threshold.

The post custirsen initiation threshold level of serum clusterin may be between 20 μg/mL and 75 μg/mL. The post custirsen initiation threshold level may be 20, 25, 30, 35, 40, 45, 50, 55, 60, 65, 70, or 75 μg/mL, or any level between any of these possible levels.

### Determining Serum Clusterin Levels

In the methods of the present invention, the manner in which the determination of serum clusterin is done is not critical.

One method for determining serum clusterin levels is an enzyme-linked immunoassay (ELISA). One such test is available commercially in microplate format with the BioVendor (2006) test kit. This ELISA uses two antihuman clusterin mouse monoclonal antibodies and a human serum-based calibrator. Calibrators, quality controls, and diluted samples are incubated in microtitration wells coated with the first antihuman clusterin monoclonal antibody. After a thorough wash, a biotin-labeled second antihuman clusterin monoclonal antibody is added to the wells and incubated with the immobilized antibody-clusterin complex. After a 1-h incubation and the subsequent washing step, streptavidin-horseradish peroxidase conjugate is added and incubated for 30 min. After the last washing step, the conjugate bound is allowed to react with the substrate (H₂0₂-tetramethylbenzidine). The reaction was then stopped by addition of an acid, and the absorbance of the resulting yellow product is measured spectrophotometrically at 450 nm. The absorbance is proportional to the concentration of clusterin.

### Dosage Units

Administration of custirsen can be carried out using the various mechanisms known in the art, including naked administration and administration in pharmaceutically acceptable lipid carriers. For example, lipid carriers for antisense delivery are disclosed in U.S. Pat. Nos. 5,855,911 and 5,417,978, which are incorporated herein by reference. In general, custirsen is administered by intravenous (i.v.), intraperitoneal (i.p.), subcutaneous (s.c.), or oral routes, or direct local tumor injection. In some embodiments, custirsen is administered by i.v. injection.

The amount of custirsen administered may be from 40 to 640 mg, or from 300 to 640 mg. Administration of custirsen may be once in a seven day period, 3 times a week, or more specifically on days 1, 3 and 5, or 3, 5 and 7 of a seven day period. In some embodiments, administration of the antisense oligonucleotide is less frequent than once in a seven day period. In some embodiments, administration of the antisense oligonucleotide is more frequent than once in a seven day period. Dosages may be calculated by patient weight, and therefore in some embodiments a dose range of about 1-20 mg/kg, or about 2-10 mg/kg, or about 3-7 mg/kg, or about 3-4 mg/kg could be used. This dosage is repeated at intervals as needed. One clinical concept is dosing once per week with 3 loading doses during week one of treatment. The amount of antisense oligonucleotide administered is one that has been demonstrated to be effective in human patients to inhibit the expression of clusterin in cancer cells.

A dosage unit may comprise a single compound or mixtures of compounds thereof. A dosage unit can be prepared for oral, injection, or inhalation dosage forms.

In some embodiments, custirsen may be formulated at a concentration of 20 mg/mL as an isotonic, phosphate-buffered saline solution for IV administration. In some embodiments custirsen may be supplied as a 32 mL solution containing 640 mg custirsen sodium in a single vial, or may be supplied as an 8 mL solution containing 160 mg custirsen sodium in a single vial. The drug product and active ingredient of custirsen sodium is a second-generation, 4-13-4 MOE-gapmer antisense oligonucleotide (ASO).

In some embodiments, custirsen may be added to 250 mL 0.9% sodium chloride (normal saline). In some embodiments, the dose may be administered using either a peripheral or central indwelling catheter intravenously as an infusion over 2 hours. Additionally, in some embodiments an infusion pump may be used.

In some embodiments, subjects may receive paclitaxel 200 mg/m² as a constant rate infusion on Day 1 of each of one or more 21-day treatment cycles. The amount of paclitaxel administered may be from 100-250 mg/m². The amount of paclitaxel administered may be 100 mg/m², 105 mg/m², 110 mg/m², 115 mg/m², 120 mg/m², 125 mg/m², 130 mg/m², 140 mg/m², 145 mg/m², 150 mg/m², 155 mg/m², 160 mg/m², 165 mg/m², 170 mg/m², 175 mg/m², 180 mg/m², 185 mg/m², 190 mg/m², 195 mg/m², 200 mg/m², 205 mg/m², 210 mg/m², 220 mg/m², 225 mg/m², 230 mg/m², 235 mg/m², 240 mg/m², 245 mg/m², or 250 mg/m². The duration of paclitaxel constant rate infusion may be from 1 to 3 hours, or from 3 to 6 hours. The duration of paclitaxel constant rate infusion may be 1, 1.5, 2, 2.5, 3, 3.5, 4, 4.5, 5, 5.5, or 6 hours. In some embodiments, subjects may receive IV carboplatin at a dose calculated for a target AUC of 6 mg/mL per min as a 30 minute constant rate infusion. The amount of carboplatin may be a dose calculated for a target AUC from 2-8 mg/mL per min. The amount of carboplatin may be a dose calculated for a target AUC of 2 mg/mL per min, 3 mg/mL per min, 4 mg/mL per min, 6 mg/mL per min, 7 mg/mL per min, or 8 mg/mL per min. In some embodiments paclitaxel and/or carboplatin may be administered less frequently than once every 21-days. In some embodiments paclitaxel and/or carboplatin may be administered more frequently than once every 21-days. In some embodiments the carboplatin is administered immediately following paclitaxel. In some embodiments the paclitaxel is administered immediately following the carboplatin.

In some embodiments of the invention, the amount of paclitaxel, carboplatin, or paclitaxel/carboplatin required for treatment of NSCLC is less in combination with custirsen, than would be required with a therapy comprising paclitaxel, carboplatin, or paclitaxel/carboplatin without custirsen.

In some embodiments, the amount of paclitaxel when taken together with custirsen is more effective to treat the human patient than when paclitaxel is administered alone.

In some embodiments, the amount of paclitaxel/carboplatin when taken together with custirsen is more effective to treat the human patient than when paclitaxel/carboplatin is administered alone.

In some embodiments, the amount of paclitaxel in combination with custirsen is less than is clinically effective when administered alone or without custirsen.

In some embodiments, the amount of paclitaxel/carboplatin in combination with custirsen is less than is clinically effective when administered without custirsen.

In some embodiments, the amount of paclitaxel when administered with custirsen is effective to reduce a clinical symptom of NSCLC of non-squamous histology in the human patient.

In some embodiments, a chemotherapeutic agent may be administered via an infusion control device (pump) using non-PVC tubing and connectors.

The pharmacokinetic (area under the time concentration curve [AUC]) and the pharmacodynamic effects (hematologic toxicity) of carboplatin are better predicted by glomerular filtration rate (GFR) based dosing as compared with the more traditional body surface area (BSA) dosing method. The Calvert formula provides a consistent method for determining carboplatin dosage in adults that should produce the desired degree of toxicity (Calvert et al., 1989).

The Calvert formula may be used to calculate the carboplatin dose:

Carboplatin dose (mg)=target AUC×(GFR+25)

The Cockcroft-Gault formula may be used to calculate the creatinine clearance (CrCl) (Cockcroft and Gault, 1976), which can be substituted for glomerular filtration rate (GFR) in the Calvert formula. Calculations may be based upon the serum creatinine value obtained within 72 hours prior to treatment for each cycle.

$$\frac{( 140 - {subject}^{′} s age ) \times {subject}^{′} s actual body weight in {kg}^{*}}{72 \times {subject}^{′} s serum creatinine ( in mg \text{/} dL )}$$

$${}^{*} For females , multiply the result by 0.85$$

In some embodiments doses of both chemotherapeutic agents may be based on the subject's actual body weight within 3 days prior to treatment. The same weight measurement may be used to calculate the dosage of both drugs.

In some embodiments, subjects may receive docetaxel 75 mg/m² as an infusion on Day 1 of each of one or more 21-day treatment cycles. The amount of docetaxel administered may be from 25 mg/m² to 100 mg/m². The amount of docetaxel administered may be about 25 mg/m², 30 mg/m², mg/m², 40 mg/m², 45 mg/m², 50 mg/m², 55 mg/m², 60 mg/m², 65 mg/m², 70 mg/m², 75 mg/m², 80 mg/m², 85 mg/m², 90 mg/m², 95 mg/m² or 100 mg/m². The duration of docetaxel infusion may be from 1 to 3 hours, or from 3 to 6 hours. The duration of docetaxel constant rate infusion may be 1, 1.5, 2, 2.5, 3, 3.5, 4, 4.5, 5, 5.5, or 6 hours. In some embodiments, docetaxel infusion is constant rate infusion.

In some embodiments, subjects may receive a taxane 75 mg/m² as an infusion on Day 1 of each of one or more 21-day treatment cycles. In some embodiments, subjects may receive a taxane 200 mg/m² as an infusion on Day 1 of each of one or more 21-day treatment cycles. The amount of taxane administered may be from 25 mg/m² to 250 mg/m². The amount of taxane administered may be about 25 mg/m², 30 mg/m², 35 mg/m², 40 mg/m², 45 mg/m², 50 mg/m², 55 mg/m², 60 mg/m², 65 mg/m², 70 mg/m², 75 mg/m², 80 mg/m², 85 mg/m², 90 mg/m², 95 mg/m² 100 mg/m², 105 mg/m², 110 mg/m², 115 mg/m², 120 mg/m², 125 mg/m², 130 mg/m², 140 mg/m², 145 mg/m², 150 mg/m², 190 mg/m², 195 mg/m², 200 mg/m², 205 mg/m², 210 mg/m², 220 mg/m², 225 mg/m², 230 mg/m², 235 mg/m², 240 mg/m², 245 mg/m², or 250 mg/m². The duration of taxane infusion may be from 1 to 3 hours, or from 3 to 6 hours. The duration of taxane constant rate infusion may be 1, 1.5, 2, 2.5, 3, 3.5, 4, 4.5, 5, 5.5, or 6 hours. In some embodiments, taxane infusion is constant rate infusion.

In some embodiments of the invention, the amount of a taxane, a platinum-based chemotherapeutic, or a combination of both required for treatment of NSCLC is less in combination with custirsen, than would be required with a therapy comprising a taxane, a platinum-based chemotherapeutic, or a combination of both without custirsen.

In some embodiments of the invention, the amount of docetaxel, platinum-based chemotherapy or docetaxel/platinum-based chemotherapy required for treatment of NSCLC is less in combination with custirsen, than would be required with a therapy comprising docetaxel, platinum-based chemotherapy or docetaxel/platinum-based chemotherapy without custirsen.

In some embodiments of the invention, the amount of a taxane required for treatment of NSCLC is less in combination with custirsen, than would be required with a therapy comprising a taxane without custirsen.

In some embodiments of the invention, the amount of docetaxel required for treatment of NSCLC is less in combination with custirsen, than would be required with a therapy comprising a taxane, a platinum-based chemotherapeutic, or a combination of both without custirsen.

In some embodiments, the amount of taxane when taken together with custirsen is more effective to treat the human patient than when a taxane is administered alone.

In some embodiments, the amount of docetaxel when taken together with custirsen is more effective to treat the human patient than when docetaxel is administered alone.

In some embodiments, the amount of a taxane/platinum-based chemotherapy when taken together with custirsen is more effective to treat the human patient than when a taxane/platinum-based chemotherapy is administered alone.

In some embodiments, the amount of docetaxel/platinum-based chemotherapy when taken together with custirsen is more effective to treat the human patient than when docetaxel/platinum-based chemotherapy is administered alone.

In some embodiments, the amount of a taxane in combination with custirsen is less than is clinically effective when administered alone or without custirsen.

In some embodiments, the amount of docetaxel in combination with custirsen is less than is clinically effective when administered alone or without custirsen.

In some embodiments, the amount of a taxane/platinum-based chemotherapy in combination with custirsen is less than is clinically effective when administered without custirsen.

In some embodiments, the amount of docetaxel/platinum-based chemotherapy in combination with custirsen is less than is clinically effective when administered without custirsen.

In some embodiments, the amount of a taxane when administered with custirsen is effective to reduce a clinical symptom of NSCLC of non-squamous histology in the human patient.

In some embodiments, the amount of docetaxel when administered with custirsen is effective to reduce a clinical symptom of NSCLC of non-squamous histology in the human patient.

In some embodiments, the amount of a platinum-based chemotherapeutic agent when taken together with custirsen is more effective to treat the human patient than when the platinum-based chemotherapeutic agent is administered alone.

In some embodiments, the amount of a platinum-based chemotherapeutic agent in combination with custirsen is less than is clinically effective when administered alone or without custirsen.

In some embodiments, the amount of a platinum-based chemotherapeutic agent when administered with custirsen is effective to reduce a clinical symptom of NSCLC of non-squamous histology in the human patient.

According to an aspect of the invention, there is provided a custirsen-containing pharmaceutical composition packaged in dosage unit form, wherein the amount of custirsen in each dosage unit is 640 mg. Said pharmaceutical composition may include a taxane and/or platinum-based chemotherapeutic agent, and may be in an injectable solution or suspension, which may further contain sodium ions.

According to an aspect of the invention, there is provided a custirsen-containing pharmaceutical composition packaged in dosage unit form, wherein the amount of custirsen in each dosage unit is 640 mg. Said pharmaceutical composition may include paclitaxel and/or carboplatin, and may be in an injectable solution or suspension, which may further contain sodium ions.

According to an aspect of the invention, there is provided a custirsen-containing pharmaceutical composition packaged in dosage unit form, wherein the amount of custirsen in each dosage unit is 640 mg. Said pharmaceutical composition may include docetaxel, and may be in an injectable solution or suspension, which may further contain sodium ions.

According to another aspect of the invention, there is provided the use of custirsen and a taxane and/or a platinum-based chemotherapeutic agent in the manufacture of a medicament for the treatment of cancer, where the medicament is formulated to deliver a dosage of 640 mg custirsen to a patient. The medicament may contain sodium ions, and/or be in the form of an injectable solution.

According to another aspect of the invention, there is provided the use of custirsen and paclitaxel and/or carboplatin in the manufacture of a medicament for the treatment of cancer, where the medicament is formulated to deliver a dosage of 640 mg custirsen to a patient. The medicament may contain sodium ions, and/or be in the form of an injectable solution.

According to another aspect of the invention, there is provided the use of custirsen and docetaxel in the manufacture of a medicament for the treatment of cancer, where the medicament is formulated to deliver a dosage of 640 mg custirsen to a patient. The medicament may contain sodium ions, and/or be in the form of an injectable solution.

General techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications Drugs and the Pharmaceutical Sciences, Vol (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol. 40 (Gilbert S. Banker, Christopher T. Rhodes, Eds.). These references in their entireties are hereby incorporated by reference into this application.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Example 1

### Clinical Trial (Phase III)—Assessment of Paclitaxel/Carboplatin in Combination with Custirsen in Preventing Progression of Non-Squamous NSCLC

A multinational, randomized, open-label phase III study comparing a standard first-line paclitaxel/carboplatin chemotherapy regimen to paclitaxel/carboplatin in combination with custirsen (TV-1011) is conducted to evaluate the safety, tolerability and efficacy in subjects with Stage IV non-squamous NSCLC.

### Study Title

A Multinational, Randomized, Open-Label Phase III Study Comparing a Standard First-Line Paclitaxel/Carboplatin Chemotherapy Regimen to Paclitaxel/Carboplatin in Combination with Custirsen (TV-1011) in Subjects with Stage IV Non-Squamous Non-Small Cell Lung Cancer.

### Treatment Duration

Subjects randomized to the custirsen arm first receive three doses of custirsen in a 5 to 9 day loading dose period prior to Day 1 of Cycle 1. Subjects randomized to both study arms have 21-day chemotherapy cycles until disease progression, unacceptable toxicity, or completion of 6 cycles; however, subjects randomized to the custirsen arm also receive weekly doses of custirsen starting at Day 1 of each of the 21-day chemotherapy cycles until disease progression, unacceptable toxicity, or completion of all 6 cycles.

### Study Population

Stage IV NSCLC of non-squamous histology.

### Study Objectives

The primary objective is to evaluate the overall survival benefit of adding custirsen to standard paclitaxel/carboplatin chemotherapy.
The secondary objective is to evaluate the effect of adding custirsen to standard paclitaxel/carboplatin chemotherapy on the rate of progression free survival (PFS) at 14 weeks.
Additional objectives are:
   To evaluate the safety and tolerability of adding custirsen to standard paclitaxel/carboplatin chemotherapy.
   To assess the effect of adding custirsen to standard paclitaxel/carboplatin chemotherapy on quality of life parameters.
   To assess the effect of adding custirsen to standard paclitaxel/carboplatin chemotherapy on serum clusterin pharmacodynamics.
   To explore the relationship between serum clusterin as a biomarker and for evaluating efficacy measures, including overall survival.
   To evaluate the effect of adding clustirsen to standard paclitaxel/carboplatin chemotherapy on other disease parameters such as progression free survival and time to progression.
   To assess the exposure of the study population to custirsen.
   To establish if custirsen alters the pharmacokinetics of paclitaxel/carboplatin.

### Study Design Overview

This is a randomized, open-label, sponsor-blinded multinational trial. Treatment consists of paclitaxel/carboplatin/custirsen vs. paclitaxel/carboplatin, which compose the two arms of the study.

After a screening period of up to 28 days, subjects are randomly assigned with equal probability to the two arms. Stratified randomization is used in order to minimize between-arm imbalance over four stratification factors: Gender, Eastern Cooperative Oncology Group (ECOG) performance status (0 vs. 1), Smoking status (former/current smoker vs. never-smoker) and Geographical Region (North America, Europe and Southeast Asia).

Subjects randomized to the custirsen arm have a 5 to 9 day loading dose period prior to Day 1 of Cycle 1. Subjects receive paclitaxel/carboplatin on a 3-week cycle either alone or with weekly custirsen infusions, until disease progression, unacceptable toxicity or completion of 6 cycles. Subjects who are removed from study treatment for any reason other than disease progression or death are followed for documented disease progression. Once disease progression is documented, subjects enter a survival follow-up phase during which data is collected regarding further cancer treatment and their survival status.

Tumor response to study treatment and disease progression is based on the criteria proposed by the Response Evaluation Criteria in Solid Tumors (RECIST) 1.1 guidelines. All subjects undergo CT or MRI scans of the chest and upper abdomen, as well as any other areas clinically indicated, at screening, then every 6 weeks, starting at week 8 for the first 26 weeks (week 8, 14, 20 and 26) and then every 12 weeks after the week 26 scan until disease progression. These time points are kept within a window of +one week, regardless of the treatment schedule. The week 26 scan is performed as scheduled, regardless of whether the patient is still in the treatment period (i.e. due to treatment delays) or has already completed the end of treatment visit. Once a patient is discontinued from study treatment for documented disease progression, scanning is no longer required. Assessment of these scans is carried out in a blinded fashion, by a Central Imaging Lab.

Note: CT scans are preferred; however, MRIs can be used for disease assessment as long as they are consistently performed for an individual subject's assessments.

Note: CT scans are performed with cuts of 5 mm or less in contiguous slice thickness.

Adverse events are recorded at each visit during the study and 28 days after the last dose of study treatment. Medical history is assessed at screening and an electrocardiogram is performed. Physical examination, assessment of the ECOG performance status, vital signs and laboratory evaluations are conducted at screening and throughout the study. Adverse events are recorded from when a subject is signed the Informed Consent Form and throughout the study, until the end of the treatment visit (28 days following last dose). They are reviewed and updated at each subsequent visit and during any phone contact with the subject.

The general health status, as reported by the subjects is assessed by the EuroQoL (EQ-5D) and FACT-L questionnaires. Medical resource utilization is also compared between the treatment arms. The EQ-5D is a standardized instrument for use as a measure of health outcome. Applicable to a wide range of health conditions and treatments, it provides a simple descriptive profile and a single index value for health status that can be used in the clinical and economic evaluation of health care as well as population health surveys. EQ-5D is designed for self-completion by subjects. In this study the instrument is self-administrated.

Pharmacodynamic blood samples for serum clusterin are drawn, in order to evaluate the arm-specific levels of serum clusterin and explore whether there is an association with efficacy measures. All serum clusterin testing is done at a central laboratory.

Pharmacokinetic testing for blood levels of custirsen, paclitaxel and carboplatin (Arm A), or paclitaxel and carboplatin (Arm B), are performed. These samples allow further exploration of the pharmacokinetic profile of custirsen, to investigate the interaction between custirsen and the paclitaxel/carboplatin combination, and to model the relationship within the paclitaxel/carboplatin/custirsen treatment arm, between exposure to custirsen (i.e., serum custirsen levels at the end of custirsen infusion) and outcome measures (e.g., clinical efficacy and toxicity parameters).

### Number of Subjects

Approximately 950 subjects with Stage IV non small cell lung cancer of non-squamous histology (NSCLC).

### Inclusion/Exclusion Criteria

### Inclusion Criteria

1. Subjects must have a histologically or cytologically confirmed diagnosis of NSCLC of non-squamous (adenocarcinoma, large cell or other) histology.
2. Males or females 18 years of age at screening.
3. Stage IV disease (according to the IASLC 7^{th} edition TNM staging, thus including patients with pleural effusion who were previously classified as Stage IIIB) that is not amenable to either surgery or radiation therapy of curative intent.
4. Life expectancy of >12 weeks.
5. Subjects must have at least one lesion meeting RECIST 1.1 criteria for measurable disease.
6. All of the following if patient has had prior radiation therapy:
   Lesion(s) used for determination of response was not previously irradiated or has increased in size (by at least 30% in the longest diameter) since the completion of radiotherapy.
   Radiotherapy to lesion(s) used for determination of response was completed at least 6 weeks prior to randomization; radiotherapy to other sites was completed at least 4 weeks prior to randomization.
7. ECOG performance status of 0 or 1.
8. Have adequate bone marrow reserve as defined by:
   Absolute neutrophil count (ANC)≧1.5×10⁹/L
   Platelets≧100×10⁹/L
   Hemoglobin≧9 g/dL
9. Have adequate liver function as defined by:
   Bilirubin≦1.5×ULN (unless elevated secondary to conditions such as Gilbert's disease)
   AST and ALT≦3×ULN 5×ULN in subjects with liver metastases at screening)
10. Have adequate renal function as defined by creatinine clearance ≧50 mL/min per the Cockcroft and Gault formula.
11. At randomization, at least 4 weeks have passed since prior major surgery.
12. At randomization, at least 4 weeks (or 5 elimination half-lives of study agent, whichever is longer) have passed since receiving any investigational agent.
13. Women of child-bearing potential practice a highly effective method of birth control during and for 3 months after the treatment period.
14. Male partners of women of child-bearing potential can be either surgically sterile, or ensure that their female partner utilizes a highly effective contraceptive method during and for 3 months after the treatment period.
15. Subjects give written informed consent prior to any protocol-specific procedures being performed and comply with the protocol requirements for the duration of the study.

### Exclusion Criteria

1. Subjects with NSCLC that is predominantly (>50%) of squamous histology.
2. Subjects that received any prior systemic anti-cancer therapy (approved or experimental) for advanced NSCLC. Subjects who have received adjuvant chemotherapy are eligible if the last administration of the prior adjuvant regimen occurred at least 1 year prior to randomization.
3. Subjects with brain metastases that are symptomatic or require ongoing treatment with steroids or anticonvulsants. Brain imaging is required for symptomatic patients to rule out brain metastases, but is not required in asymptomatic patients.
4. Subjects with an active second malignancy (except in situ carcinoma of the cervix, adequately treated non-melanomatous skin cancers, clinically localized prostate cancer, superficial bladder cancer or other malignancy treated at least 2 years previously with no evidence of recurrence).
5. Subjects with persistent grade 2 or greater toxicity related to prior therapy (except alopecia or anemia).
6. Subjects with grade 2 or greater peripheral neuropathy.
7. Medical conditions such as heart failure, myocardial infarction, uncontrolled hypertension, uncontrolled diabetes mellitus, cerebrovascular accident or acute hepatitis within 3 months of randomization or treatment of a major active infection within 1 month of randomization, an ongoing serious cardiac arrhythmia requiring medication as well as any other significant concurrent medical illness that in the opinion of the Investigator would preclude protocol therapy.
8. Planned concomitant participation in another clinical trial of an experimental agent, vaccine, or device. Concomitant participation in observational studies is acceptable.
9. Female subjects who are pregnant or breastfeeding.
10. Subjects with a known sensitivity to any component of paclitaxel or carboplatin.

### Dosage and Route of Administration

### Study Agent: Custirsen Sodium

640 mg in 250 mL normal saline IV over 2 hours.

### Chemotherapy: Paclitaxel and Carboplatin

Paclitaxel 200 mg/m² IV over 3 hours. Carboplatin AUC 6.0 mg/ml/min IV over 30 minutes.

### Custirsen Loading Dose Period

If randomized onto the investigational arm (Arm A), subjects receive custirsen. The schedule of administration of custirsen starts with a Loading Dose Period. The first dose of custirsen for the Loading Dose Period is administered within 4 days following randomization.

Three doses of 640 mg custirsen are administered IV during the Loading Dose Period (Days −9 to −1). There is at least one “non-infusion” day between each administration of custirsen (i.e. every other day) during the Loading Dose Period and between the third loading dose of custirsen and Day 1 of Cycle 1. The day prior to Day 1 of Cycle 1 (Day 0) is a “no treatment” day. There are no more than 4 days between the last loading dose and Day 1 of Cycle 1. A common schedule is to give the three loading doses of custirsen on Monday, Wednesday and Friday with Day 1, Cycle 1 starting on the following Monday. Up to nine days are allowed for completing the three loading doses prior to Day 0 to account for clinic visits, weekends, and holidays. Subjects receive each dose of custirsen as a 2 hour infusion. Because grade 1-2 constitutional symptoms (e.g., fever and chills) are seen in 50-60% of subjects during the first two to three custirsen infusions, all subjects are be premedicated with ibuprofen (400 mg) or acetaminophen (500-1000 mg) 30-60 minutes prior to and every 4-6 hours for 24 hours following each of the three loading doses of custirsen during the Loading Dose Period only.

### 21 Day (Three-Week) Treatment Cycles

### Arm A Subjects Only:

Following completion of the loading dose period, and within a maximum of 4 days, 640 mg custirsen is given IV weekly on Days 1, 8, and 15 of each 21 day cycle. Custirsen is administered prior to paclitaxel and carboplatin on Day 1 of each cycle.

### Arm B Subjects Only:

The first doses of paclitaxel and carboplatin are administered within 4 days following randomization.

### Both Arm A and Arm B Subjects:

Paclitaxel (200 mg/m²) and Carboplatin AUC 6.0 mg/ml/min IV is administered IV on Day 1 of each 21 day cycle.

Treatment cycles continue until disease progression, unacceptable toxicity, or completion of 6 cycles.

### Dose Modifications for Toxicity

Toxicities are graded using the NCl CTCAE, Version 4.0.

In general, the need for dose modifications is assessed based on laboratory values or physical signs obtained within 72 hours prior to treatment on Day 1 of each cycle. While dose reductions are employed for paclitaxel and carboplatin, custirsen is always given at the 640 mg dose, but the dose may be withheld if necessary. If Day 1 chemotherapy is delayed for hematological toxicity due to paclitaxel or carboplatin, weekly custirsen administration continues, unless the criteria for holding custirsen are met (herein below). Treatment may be delayed no more than three weeks to allow recovery from toxicity. If a subject has greater than a 3-week lapse in study treatment for any reason, the subject has an “End of Treatment” assessment and enters the “Off-Treatment Follow-up Period” until disease progression.

The dose of paclitaxel or carboplatin is not re-escalated once the dose is reduced. If more than two dose reductions of paclitaxel or carboplatin are required, the subject is removed from study treatment. If custirsen, paclitaxel or carboplatin is discontinued, the subject is removed from study treatment. These subjects have an “End of Treatment” assessment and enter the “Off-Treatment Follow-up Period” until disease progression.

The reason for modifying the dose of any study treatment (custirsen, paclitaxel and/or carboplatin) is recorded.

### Specific Dose Levels for Paclitaxel and Carboplatin Modification

The table below defines the specific dose level modifications for paclitaxel and carboplatin.

TABLE 1
Dose Modification Levels for Paclitaxel and Carboplatin

| | Paclitaxel | Carboplatin-Target |
|---|---|---|
| Dose Level | (mg/m²) | AUC (mg/ml/min) |
| 100% | 200 | 6 |
| First dose reduction | 175 | 5 |
| Second dose reduction* | 150 | 4 |

*If more than two dose reductions of paclitaxel or carboplatin are required, the subject is removed from study treatment.

### Hematologic Toxicity

G-CSF and other growth factors are allowed to assist in subject management. The following section delineates how to modify or hold the dose of paclitaxel and carboplatin based on the hematology results and clinical findings on Day 1 of each cycle.

If Day 1 chemotherapy is delayed for hematological toxicity, weekly custirsen administration continues, unless the criteria for holding custirsen have been met.

No dose reductions are required for anemia. Subjects may be supported with transfusions or erythropoietin to maintain their hematocrit at acceptable levels.

Prior to receiving each cycle of therapy, subjects must have an absolute neutrophil count (ANC) ≧1.5×109 cells/L and PLT≧100×109 cells/L. Treatment may be delayed no more than three weeks to allow for hematologic recovery. If ANC <1.5×109 cells/L and/or PLT<100×109 cells/L after 3 weeks, chemotherapy is discontinued.

If on Day 1 of a cycle, the ANC is <1.5×109 cells/L and/or the platelet count is <100×109 cells/L, both paclitaxel and carboplatin are withheld. Blood counts are repeated weekly. Once the ANC recovers to ≧1.5×109 cells/L and the platelet count is ≧100×109 cells/L, treatment with paclitaxel and carboplatin is resumed. If this lasts for more than one week, chemotherapy doses are reduced by 1 dose level.

Chemotherapy doses are also reduced by 1 dose level if at any time during the previous cycle for one of the following has occurred:
Grade 3 febrile neutropenia (defined as an ANC <1.0×109 cells/L and a temperature >38.5° C.)
Documented infection with grade 3 neutropenia (defined as an ANC <1.0×109 cells/L)
Grade 4 neutropenia (defined as an ANC <0.5×109 cells/L) lasting 7 days or more
Grade 4 thrombocytopenia (platelet count<25×109/L) lasting 7 days or more

In any of the above four cases, weekly custirsen infusions are also held, and are resumed at the full 640 mg dose, once the toxicity recovers to grade 2 or less.

When a chemotherapy dose reduction is required at the beginning of a 21-day cycle, paclitaxel and carboplatin are reduced together and no dose re-escalation is permitted in future cycles.

If any of the following occur, the subject is removed from study treatment:
Grade 4 febrile neutropenia or grade 4 infection with neutropenia
Thrombocytopenic hemorrhage (gross not occult bleeding) associated with a platelet count <50×109/L

### Hepatic Toxicity

The LFT values (AST, ALT and bilirubin) on Day 1 of each cycle are used in determining if a dose reduction is necessary. Chemotherapy (paclitaxel and carboplatin) and custirsen are held in any case of LFT elevation (AST, ALT and/or bilirubin) to grade 3 or greater and resumed once the toxicity recovers to grade 2 or less. Subsequently, the dose of paclitaxel is reduced by 1 dose level in the next cycle. Treatment with carboplatin and custirsen is resumed at the full dose. If treatment is withheld, LFT values must recover within 3 weeks or the subject's protocol treatment is discontinued.

### Renal Toxicity

To enter the trial, subjects are required to have adequate renal function as defined by creatinine clearance ≧50 ml/min per the Cockcroft and Gault formula.

For on study decrease in the creatinine clearance of up to 30 ml/min (Grade 2), the carboplatin dose is adjusted according to the Calvert formula. Paclitaxel and custirsen are continued at the full dose.

For grade 3 decrease in creatinine clearance (to below 30 ml/min) or grade 3 increase in creatinine (>3× baseline value or >4.0 mg/dl), all protocol treatment is withheld until toxicity resolves to ≦grade 2. Upon resolution to ≦grade 2, paclitaxel and carboplatin are resumed at a dose reduction of one level, and custirsen at the full dose.

If the toxicity is not resolved within 3 weeks, the subject is discontinued from study treatment. If the toxicity recurs in a subsequent cycle at a grade 3 or higher, the subject is removed from study treatment and followed for disease progression.

For any grade 4 renal toxicity (defined as creatinine clearance <15 ml/min or dialysis or renal transplant indicated) the subject is removed from protocol treatment.

### Paclitaxel and Carboplatin—Dose Modifications for Neurotoxicity

For grade 4 neurotoxicity, the subject should be removed from protocol treatment.

For grade 3 neurotoxicity, paclitaxel and carboplatin are withheld until toxicity resolves to ≦grade 2. Both are then resumed at a dose reduction of one level.

Paclitaxel and Carboplatin—Dose Modifications for Diarrhea and/or Vomiting

In the case of grade 4 (life threatening) diarrhea and/or vomiting, the subject is removed from protocol treatment.

In the case of grade 3 diarrhea (≧7 stools per day over baseline; incontinence; need for IV fluids >24 hours; limiting self care ADL or hospitalization), paclitaxel and carboplatin are held until resolution to grade ≦2 and the subject receives prophylactic anti diarrhea therapy in subsequent cycles.

If grade 3 diarrhea recurs despite maximal prophylactic treatment (e.g., loperamide, diphenoxylate hydrochloride with atropine, octreotide), the subject is removed from protocol treatment.

In the case of grade 3 vomiting [>=6 episodes (separated by 5 minutes) in 24 hrs; tube feeding, TPN or hospitalization indicated], paclitaxel and carboplatin are held until resolution to ≦grade 2 and the subject receives prophylactic anti emetic therapy in subsequent cycles.

If grade 3 vomiting recurs despite maximal prophylactic treatment (e.g. ondansteron, metoclopramide, dexamathasone) the subject is removed from protocol treatment.

### Paclitaxel—Cardiovascular Toxicity

Cardiac rhythm disturbances have occurred infrequently in subjects receiving paclitaxel. Most subjects were asymptomatic and cardiac monitoring is not required. Transient asymptomatic bradycardia has been noted in as many as one third of subjects. More significant atrioventricular (AV) block has rarely been noted. Cardiac events should be managed as follows:
Asymptomatic bradycardia—no treatment required
Asymptomatic AV block or any symptomatic arrhythmia during infusion—paclitaxel infusion is stopped, arrhythmia is managed according to standard practice. All protocol treatment is discontinued.
Chest pain and/or symptomatic hypotension (below 90/60 mmHg or requiring fluid replacement)—paclitaxel infusion is stopped. An ECG is performed. The pation is given intravenous diphenhydramine and dexamethasone as above if hypersensitivity is considered. Also, epinephrine or bronchodilators are considered if chest pain is not thought to be cardiac. All protocol treatment is discontinued.

### Paclitaxel—Allergic Reaction/Hypersensitivity

Subjects who had a mild to moderate hypersensitivity reaction have been successfully rechallenged with paclitaxel, but careful attention to prophylaxis and bedside monitoring of vital signs is recommended.

Mild symptoms: Complete paclitaxel infusion. Supervise at bedside. No treatment required.

Moderate to severe symptoms (grade 2 or 3): Paclitaxel infusion is stopped. Diphenhydramine 25-50 mg and intravenous dexamethasone 10 mg is given. Paclitaxel infusion is resumed after recovery of symptoms at a low rate, 20 mL/hour for 15 minutes, then 40 mL/hour for 15 minutes, then, if no further symptoms, at full dose rate until infusion is complete. If symptoms recur, paclitaxel infusion is stopped and protocol treatment is discontinued. Subjects who experience grade 3 hypersensitivity to paclitaxel receive twice the dose of steroid premedication for subsequent cycles. Paclitaxel administration is slower (half the usual rate) for the first hour of the infusion. The infusion rate is increased during the later 2 hours. The total duration of paclitaxel infusion remains unchanged, i.e. 3 hours.

Severe life-threatening symptoms (grade 4, anaphylaxis): Paclitaxel infusion is stopped and subject is given IV diphenhydramine and dexamethasone as herein above. Epinephrine or bronchodilators are added if indicated protocol treatment is discontinued.

### Dose Modifications for Non-Hematological Grade 3 or 4 Toxicities

For any grade 4 NCl CTCAE denoted as “life threatening” toxicity, the subject is discontinued from study treatment and followed for disease progression.

For any grade 3 or 4 event defined below (Note: this does not include alopecia, nausea, cough, headache, insomnia, nail changes, changes in taste and nonsymptomatic laboratory values [e.g., sodium, potassium, magnesium]), paclitaxel, carboplatin and custirsen, are held until resolution to ≦grade 2:
Grade 4 NCl CTCAE denoted as “disabling” (tinnitus, fatigue, asthenia, lethargy, malaise, arthralgia, myalgia, dizziness, tremor) or;
Grade 3 NCl CTCAE not mentioned in the sections above and viewed by the Investigator as clinically significant

Upon resolution to ≦grade 2, both paclitaxel and carboplatin are resumed with a reduction of one dose level. If the toxicity does not resolved within 3 weeks, the subject is discontinued from study treatment. If the toxicity recurs in a subsequent cycle at a grade 3 or higher, the subject is removed from study treatment and followed for disease progression.

### Statistical Analysis

### Primary Outcome

The primary outcome measure is overall survival (OS). Time to death from any cause is the primary efficacy endpoint. The primary analysis is a stratified log-rank test (stratified by the above-identified stratification factors).

### Secondary Outcome

Progression Free Survival (PFS) at 14 weeks from randomization. For each subject, a Week 14 Progression Free Survival (PFS) status variable “Alive Without Progression (AWP)” is defined as one (1) if the subject is alive and found to be free of evidence of progression as defined herein above, and zero (0) if otherwise. The proportions of subjects in each arm for which AWP=1 are compared as a secondary analysis of efficacy. The Cochran-Mantel-Haenszel test with the above-defined stratification factors is used for testing.

### Results

The combination treatment of custirsen and paclitaxel/carboplatin administered to arm A subjects is safe and well tolerated, with an acceptable adverse events profile. Arm A subjects (custirsen+paclitaxel/carboplatin) have prolonged survival compared to Arm B subjects (paclitaxel/carboplatin). Additionally, progression free survival is increased in Arm A subjects and a statistically significant higher proportion of Arm A subjects survive free of progression for at least 14 weeks compared to Arm B subjects. Overall progression free survival is improved in arm A subjects. One or more symptoms of NSCLC, such as chest pain, pleural effusions, pulmonary edema, dyspnea, or hemoptysis occasionally improve in Arm A subjects compared to Arm B subjects. Furthermore, quality of life improves in Arm A subjects compared to Arm B subjects.

### Example 2

### Correlation of Serum Clusterin Levels to the Duration of Individual Survival in NSCLC

In this Example, the baseline clusterin levels in patients receiving treatment within Arm A of Example 1 are analyzed and compared to clinical outcome. A subpopulation of these patients having a baseline clusterin level below 71 μg/mL are more likely to substantially benefit from anti-clusterin therapy compared to patients with baseline levels above 71 μg/mL. Specifically, patients with baseline clusterin levels below 71 μg/mL tend to survive longer than patients with baseline clusterin levels above 71 μg/mL. These data fit with a previous study (described herein below) that indicated a predictive threshold level of baseline clusterin in patient serum. Patients with baseline clusterin levels below this threshold were likely to benefit more from anti-clusterin therapy than patients with levels above the threshold.

The relationship between serum clusterin levels and the duration of individual survival was evaluated during a phase 1-2 study of weekly custirsen plus a gemcitabine/platinum-based regimen in patients with stage IIIB or IV NSCLC. Not all of the patients in this study had baseline levels of clusterin measured. However, starting with N=69 subjects with measured baseline clusterin levels, fitting the Cox proportional hazards (PH) model with baseline clusterin as sole predictor gives p=0.10; the coefficient is positive, so that higher clusterin is associated with increased survival risk. Among the N=55 subjects who also had post-baseline data collected, fitting the same PH model gives p=0.05, again with positive coefficient.

Splitting baseline clusterin levels into Low and High strata yielded a dichotomous predictor more effective than the measured clusterin levels. A careful search for possible cutpoints, using all N=69 subjects with baseline clusterin levels, produced 71 μg/mL as a strong candidate.

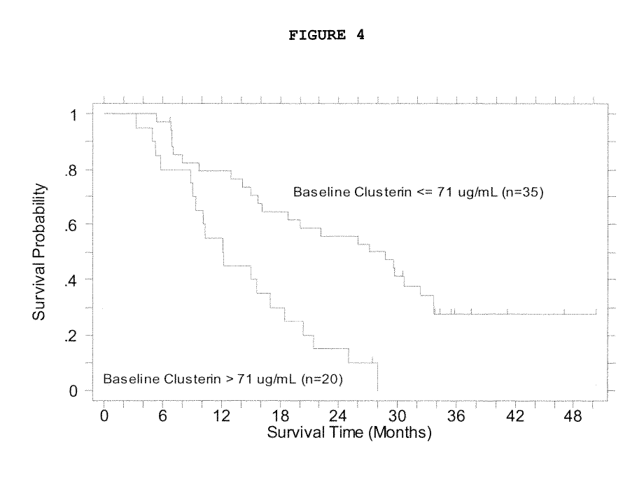

Among all N=69 subjects with a baseline clusterin level, there were N=45 with clusterin ≦71 μg/mL (median survival time was 18.8 months), and N=26 subjects with baseline clusterin >71 μg/mL (median survival time was 10.2 months); the log-rank p-value=0.004. Removing the N=14 early discontinuers leaves N=35 subjects with baseline clusterin ≦71 μg/mL (median survival time was 28.7 months), and N=20 subjects with baseline clusterin >71 μg/mL (median survival time was 12.2 months); the log-rank p-value=0.0002. FIG. 4 shows the Kaplan-Meier curves corresponding to the 71 μg/mL cutpoint.

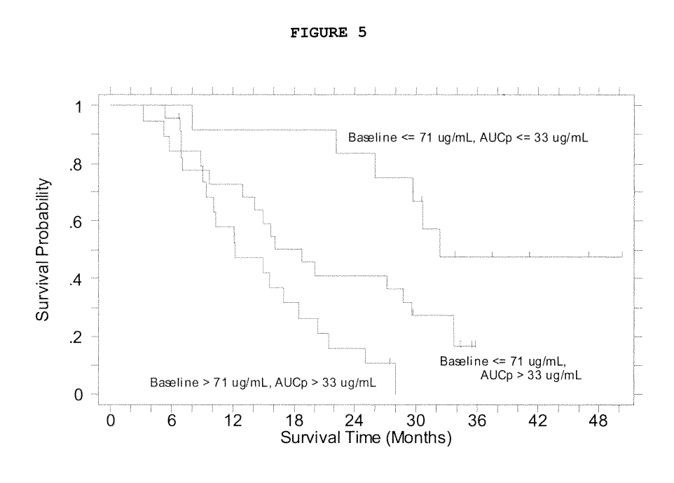

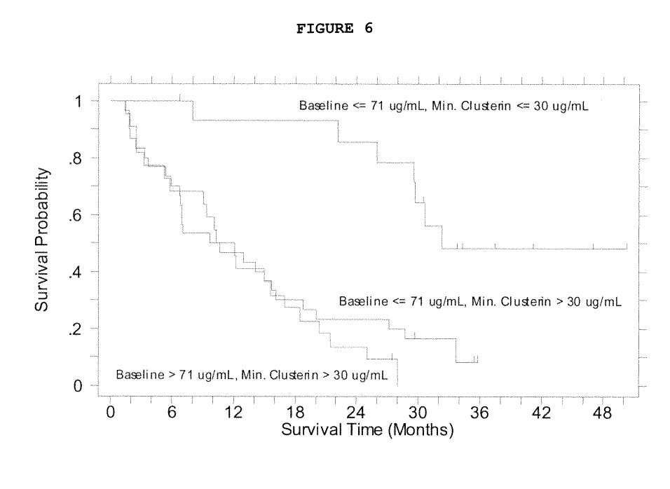

To further assess the relevance of baseline clusterin as a predictor of survival, data from the study were further segregated based on average and minimum clusterin levels achieved during treatment. FIG. 5 shows the Kaplan-Meier curves corresponding to the 71 μg/mL cutpoint for baseline clusterin and a 33 μg/mL cutpoint for average clusterin. FIG. 6 shows the Kaplan-Meier curves corresponding to the 71 μg/mL cutpoint for baseline clusterin and a 30 μg/mL cutpoint for minimum clusterin. In both cases, the combination of low baseline clusterin and low levels during therapy provided the greatest probability of extended survival.

It is surprising that similar results are observed upon treatment of a different patient population with different therapy as described in Examples 1 and 3. Additionally, one would expect that patients with more clusterin would have a greater need for, and thus benefit more from anti-clusterin therapy than patients with lower levels. Thus, the observation of a baseline threshold level below which patients receiving custirsen plus a taxane/platinum bases chemotherapy combination such as paclitaxel/carboplatin, or a taxane based chemotherapy such as docetaxel are likely to substantially benefit is an important and novel discovery.

### Example 3

### Clinical Trial (Phase III)—Assessment of Custirsen in Combination With Docetaxel Versus Docetaxel For Treatment of Lung Cancer

### Study Title

A Multinational, Randomized, Open-Label Phase III Study of Custirsen (TV-1011/0GX-011) In Combination With Docetaxel Versus Docetaxel As A Second-Line Treatment In Patients with Advanced or Metastatic (Stage IV) Non Small Cell Lung Cancer.

### Treatment Duration

Patients randomized to the custirsen arm (Arm A) have 3 doses of custirsen administered in a 5 to 9 day Loading Dose Period prior to Day 1 of Cycle 1. Patients in Arm A receive custirsen on Days 1, 8 and 15, and docetaxel on Day 1 of the 21-day cycles. Patients in Arm B receive only docetaxel on Day 1 of the 21-day cycles. Patients randomized to both arms have 21-day chemotherapy cycles until disease progression, unacceptable toxicity, withdrawal of consent or protocol specified parameters to stop treatment.

### Study Population

Patients with advanced or metastatic (Stage IV) non small cell lung cancer (NSCLC) who have received one prior line of platinum-based systemic anticancer therapy.

### Study Objectives

Primary objective:
To evaluate the benefit of the combination regimen of custirsen and docetaxel in the improving the Overall Survival (OS) of patients with advanced or metastatic (Stage IV) NSCLC who have received one prior line of platinum-based systemic anticancer therapy.

### Secondary Objectives:

### Efficacy:

To compare Progression Free Survival (PFS), Objective Response Rate (ORR), and duration of response (CR or PR), between patients receiving docetaxel with or without custirsen.
To compare the arms with respect to Quality of Life (QoL) parameters.

### Safety:

To assess the safety profile of custirsen in combination with docetaxel.

### Exploratory Objectives:

To explore clinical efficacy of custirsen (PFS, OS) in subsets of patients, according to disease parameters and genetic markers.
To model the relationship within Arm A, between exposure to custirsen (i.e., plasma custirsen levels) and outcome measures (e.g., clinical efficacy and toxicity parameters).
To explore the pharmacokinetics of custirsen.
To explore the effect of custirsen on pharmacodynamic biomarkers in the plasma samples.
To compare the arm specific levels of serum clusterin and explore whether there is an association with efficacy measures.

### Study Design Overview

This is a multinational, randomized, open-label, Phase III study in advanced or metastatic (Stage IV) NSCLC patients previously treated with first-line platinum-based therapy.

After a screening period of up to 28 days, patients are randomized 1:1 to receive either docetaxel and custirsen (Arm A) or docetaxel (Arm B). Randomization is stratified by gender (male vs. female), NSCLC histology (squamous vs. non-squamous), best overall response to the first-line platinum-based therapy (SD/CR/PR vs. PD) and ECOG PS (0 vs. 1) to minimize imbalance at randomization. Patients randomized to Arm A receive 3 doses of custirsen administered in a 5 to 9 day Loading Dose Period prior to Day 1 of Cycle 1. Following the Loading Dose period, patients in Arm A receive custirsen on Days 1, 8 and 15, and docetaxel on Day 1 of the 21-day cycle. Patients in Arm B receive only docetaxel on Day 1 of the 21-day cycle. Patients randomized to both study arms have 21-day chemotherapy cycles until disease progression, unacceptable toxicity, withdrawal from study treatment, or protocol specified parameters to stop treatment. Patients who are removed from study treatment for any reason other than disease progression or death are followed for documented radiological disease progression. All patients who discontinued study treatment are followed to collect further anticancer treatment and survival information until death, loss to follow-up, withdrawal of consent, or up to 12 months after the end of treatment visit for the last patient on the study, whichever comes first.

Tumor response to study treatment and disease progression are based on the criteria proposed by the Response Evaluation Criteria in Solid Tumors (RECIST) guideline (version 1.1). All patients undergo CT or MRI scans of the chest and upper abdomen, as well as any other areas clinically indicated, at screening, then every 6 weeks, starting at week 8 after randomization until disease progression. Tumor measurement is also performed during the end of treatment visit if it is not done within the previous 6 weeks. Patients who discontinue study treatment for reasons other than disease progression continue to have tumor measurements per RECIST v1.1 until disease progression, start of new anticancer therapy, withdrawal of consent, lost to follow-up, or death, whichever occurs first. Assessment of these scans is carried out in a blinded fashion, by a Central Imaging Lab designated by the Sponsor.

Adverse events and concomitant medications are collected throughout the study up to 28 days after the last dose of study treatment. Medical history is assessed, documented results of EGFR and KRAS mutation status are collected, if available, and an electrocardiogram is performed at screening. Physical examination, assessment of the ECOG Performance Status, vital signs, and laboratory evaluations are conducted at screening and throughout the study.

The general health status, as reported by the patients, are assessed by the Functional Assessment of Cancer Therapy—Lung (FACT-L) questionnaire.

Pharmacodynamic blood samples for serum clusterin are drawn in order to compare the arm-specific levels of serum clusterin and explore whether there is an association with efficacy measures. All serum clusterin testing is done at a central laboratory.

### Pharmacokinetic Evaluation:

A subset of patients in Arm A undergo PK sampling for custirsen level determination.

### Study Stopping Criteria:

Two formal interim analyses may stop the trial early based on inadequate evidence of clinical benefit or futility:
1. The first assessment for stopping early occurs in two steps. In the first step, PFS rate (proportion of patients alive without disease progression) at 14 weeks is analyzed after the first 170 randomized patients have the chance to complete week 14 scheduled tumor assessment and assessments are reviewed by the independent radiologist. If this first criterion shows an improvement in the PFS rate at 14 weeks based on predefined criteria (i.e. one-sided p-value≦0.1 in the Chi-square test comparing PFS rates), the trial is not stopped. However, if the required improvement in PFS rate at 14 weeks criterion is not observed, then an early survival futility analysis on the first 100 death events is performed as a second step to either stop the trial early or continue. NOTE: Study enrollment continues while the pre-defined criteria are being assessed. The second step of survival futility analysis is not performed if the first criterion assuring required improvement in PFS rate is met.
2. The second interim analysis is for futility and is performed when 50% of the death events (425 deaths) have occurred.

Blinding is maintained for all interim analyses and to the criteria leading to study continuation in the first interim analysis with the DSMC. If the study is not stopped early in the first interim analysis, up to 200 sites are activated to accelerate enrollment of 1100 patients for completion of the study.

The trial is not stopped early in order to claim efficacy.

### Inclusion/Exclusion Criteria

### Inclusion Criteria

1. Patients must have a histologically or cytologically confirmed, unresectable, advanced or metastatic (Stage IV per AJCC 7^{th} edition TNM staging) NSCLC
2. Males or females 18 years of age at screening.
3. Life expectancy of >12 weeks from screening, according to the investigator's assessment.
4. Patients must have received one prior line of platinum-based systemic anticancer therapy for advanced or metastatic NSCLC. Prior maintenance therapy is allowed and will be considered as the same line of therapy when continued without discontinuation after initiation of a treatment regimen.
5. Patients must have documented radiological disease progression either during or after the first-line therapy.
6. Patients must have at least one measurable lesion per RECIST 1.1 criteria.
7. ECOG performance status of 0 or 1 at screening.
8. Have adequate electrolyte values, bone marrow, renal and liver functions at screening as defined below:
   Absolute neutrophil count (ANC)≧1.5×109/L
   Platelets≧100×109/L
   Hemoglobin≧9 g/dL
   Serum creatinine≦1.5× upper limit of normal (ULN)
   Total Bilirubin≦1.0×ULN (unless elevated secondary to benign conditions such as Gilbert's disease)
   AST and ALT≦1.5×ULN
   Alkaline phosphatase≦2.5 ULN
   Electrolyte values (sodium, potassium and magnesium)≧1×LLN and ≦1×ULN. Patients with corrected electrolyte values are eligible
9. Resolution of any toxic effects of prior therapy to Grade ≦1 according to NCl CTCAE, v4.0 (exception of alopecia and ≦Grade 2 peripheral neuropathy).
10. Females of child-bearing potential must have negative serum pregnancy test within 72 hours before randomization.
11. Women of child-bearing potential practice a highly effective method of birth control during and for 3 months after the chemotherapy/custirsen last dose. Male partners of women of child-bearing potential can be either surgically sterile, or ensure that their female partner utilizes a highly effective contraceptive method during and for 3 months after chemotherapy/custirsen last dose.
12. Patients must be willing and able to give written informed consent prior to any protocol-specific procedures being performed and comply with the protocol requirements for the duration of the study.

### Exclusion Criteria

1. Patients treated with any systemic anti-cancer therapy for NSCLC within 21 days prior to randomization.
2. Radiotherapy 2 weeks prior to randomization. Patients must have recovered from all radiotherapy-related toxicities.
3. Major surgical procedure within 4 weeks prior to randomization. Patient must have recovered from all surgery-related complications.
4. Patients with known CNS metastases (Patients with any clinical signs of CNS metastases must have a CT or MRI of the brain to rule out CNS metastases in order to be eligible for participation in the study. Patients who have had brain metastases treated with radiotherapy or surgically removed should be clinically stable off corticosteroid treatment at least 3 weeks prior to randomization.
5. Patients with history of another active primary malignancy (except in situ carcinoma of the cervix, adequately treated non-melanomatous skin cancers, clinically localized prostate cancer, superficial bladder cancer or other malignancy treated at least years previously with no evidence of recurrence).
6. Medical conditions such as heart failure, myocardial infarction, uncontrolled hypertension, uncontrolled diabetes mellitus, cerebrovascular accident or acute hepatitis within 3 months of randomization or treatment of a major active infection within one month of randomization, as well as an ongoing cardiac arrhythmia requiring medication (≧Grade 2, according to NCl CTCAE v4.0) or any other significant concurrent medical illness that in the opinion of the Investigator would preclude protocol therapy.
7. Planned concomitant participation in another clinical trial of an experimental agent, vaccine, or device. Concomitant participation in observational studies is acceptable.
8. Female patients who are breastfeeding.
9. Patients previously treated with docetaxel for NSCLC or with a known sensitivity to taxane therapies.

### Dosage and Route of Administration

Study Agent: Three loading doses of custirsen 640 mg IV over 2 hours administered in 5 to 9 days prior to Day 1 of Cycle 1, then custirsen 640 mg IV over 2 hours on Days 1, 8 and 15 of a 21-day cycle.

Chemotherapy: Docetaxel 75 mg/m² IV over 1 hour on Day 1 of a 21-day cycle.

In Arm A, docetaxel is administered immediately after custirsen infusion.

Premedications for Custirsen During the Loading Dose Period Only (Arm A):

Ibuprofen (400 mg) is administered 30-60 minutes prior to and every 4-6 hours for 24 hours following each infusion of custirsen during the Loading Dose Period. If a patient cannot tolerate ibuprofen, acetaminophen (650 mg) is substituted for ibuprofen. Further administration of premedications following the Loading Dose Period is at the discretion of the Investigator.

### Premedication for Docetaxel Treatment:

All patients are premedicated with oral corticosteroid as described below or per local Institutional standards:

Dexamethasone 8 mg twice daily for 3 days starting 1 day prior to docetaxel administration to reduce the incidence and severity of fluid retention as well as the severity of hypersensitivity reactions.

### Concomitant Medications

Following concomitant medications considered as supportive care are acceptable while participating in this study:
Prophylactic or therapeutic use of antiemetics at investigator's discretion.
Hematopoietic growth factors should be used in accordance with the guidelines established by the American Society of Clinical Oncology (ASCO), unless otherwise specified by Institutional standards.
Palliative radiation therapy for symptomatic non-target bone lesions.
Anticoagulation therapy is allowed and is at the discretion of the Investigator.
Standard nonsurgical therapies for concurrent medical conditions.
Short-term treatment with high doses of corticosteroids is permitted for the treatment of COPD or other inflammation exacerbation.

Following concomitant therapies are not permitted while participating in this trial:
Other investigational agents.
Any concurrent anticancer therapy including chemotherapy, radiotherapy for target lesions, surgery, hormonal therapy, or immunotherapy.
Docetaxel is a CYP3A4 substrate. Concomitant use of docetaxel and drugs that inhibit CYP3A4 may increase exposure to docetaxel and should be avoided.

### Outcome Measures:

### Primary Efficacy Variable and Endpoint:

The primary endpoint and variable for the study is overall survival (OS), defined as the time from date of randomization to the date of death from any cause.

### Secondary Efficacy Variables and Endpoints:

Progression Free Survival (PFS), defined as the time from the date of randomization to the first objective documented progression per RECIST v1.1 or death due to any cause, whichever occurs first.

The Objective Response (OR) is defined as achieving a best overall response of complete response (CR) or partial response (PR), as defined using RECIST v1.1.

The duration of overall response (CR or PR) is defined as the time from the first occurrence of CR or PR until the date of the first documented disease progression (taking as reference for progressive disease the smallest measurements recorded on study) or death.

### Quality of Life Parameters: Functional Assessment of Cancer Therapy—Lung (FACT-L)

### Safety Variables:

Occurrence of Adverse events throughout the study
Clinical laboratory test results
Vital signs and body weight measurements

### Pharmacokinetic Variables:

Custirsen levels for population PK analysis

### Statistical Considerations:

### Sample Size

The maximal sample size for this study was calculated based on OS for final analysis. This size depends on the number of death events required. To detect a hazard ratio (HR) of 0.8 at one-sided significance level (alpha) of 0.025 and power of 90%, a total of 850 death events are required. Based on assumption of exponential survival time distribution with median survival time of 9 months in the control arm, recruitment period of 48 months (assumed recruitment rate of 0.18 patients per site per month, starting with about 70 sites and increasing to about 200 sites), with additional 8 months of follow-up, the required sample size is 1100 patients (550 per arm). The calculation of target events was performed using EAST software, taking into account the futility analysis at 50% of events.

### Sample Size and Timing for 1st Interim Assessment

Sample size for the alive without progression (AWP) at week 14 analysis was determined at 170 evaluable patients (85 patients per arm). Sample size for the early OS futility analysis was determined at 100 death events, corresponding to randomization of approximately 235 patients. Specification of the rules for stopping the study early and associated false-positive (go decision despite no difference) and false-negative (stopping the trial despite true benefit) probabilities are provided in the Statistical Analysis Plan and are detailed in the operation characteristics section below. Based on recruitment rates and exponential survival time distribution for the control arm with median of 9 months (as specified above), realization of 100 death events occurs at about 19-20 months from beginning of randomization. Completion of week 14 progression assessment for the first 170 enrolled patients occurs at approximately 18.5 months after beginning of randomization. Both analyses may be done together since the PFS assessment (alive without event) is centrally reviewed before the futility assessment and death events can be assessed without delay.

### Multiple Comparisons and Multiplicity

In this Phase III study, there may be only one analysis for demonstrating efficacy. The efficacy analysis is performed once the pre-defined target of 850 death events is realized, using type 1 error probability of one-sided 0.025.

Two formal interim analyses for stopping the trial early are based on pre-defined assessments for inadequate evidence of clinical benefit or futility. As these analyses indicate action regarding the trial only if results are found that indicate the investigational treatment is inadequate or futile, no adjustment to type 1 error for the final analysis is made. The trial is not stopped early in order to claim efficacy.

The secondary efficacy objectives are relevant to regulatory goals only if there is success with respect to the primary objective. The secondary efficacy analysis is tested using one-sided 0.025, according to their hierarchy, provided that the primary efficacy analysis is significant. There are no further considerations of multiplicity and additional test results are considered exploratory.

### Primary Efficacy Variable Analysis

All patients randomized into this trial are included in the primary analysis, according to the randomized arm (“intent-to-treat” analysis). A patient that is not reported as dead before data cut-off or has dropped out from survival follow-up is censored at his last known alive date. The primary analysis is a stratified log-rank test (stratified by the previously identified stratification factors). Hazard ratio and its 95% confidence interval (CI) are estimated using a stratified Cox propotional hazards model (stratified by the previously identified stratification factors). Kaplan-Meier plot are used to display estimated survival probabilities.

### Randomization

Patients are stratified before randomization as described above. Centralized Randomization are performed using blocks with a 1:1 ratio into the 2 treatment arms. Randomization is stratified by gender (male vs. female), NSCLC histology (squamous vs. non-squamous), best overall response to the first-line platinum-based therapy (SD/CR/PR vs. PD) and ECOG PS (0 vs. 1) to minimize imbalance at randomization.

### 1. Operating Characteristics of 1st Interim Assessment for Inadequate Evidence of Clinical Benefit or Futility

### Step 1: Binary Progression Assessment at 14 Weeks.

The statistical rule was chosen to provide 10% false-positive probability, if in fact there is no difference in progression rates at Week 14 between the two arms, using 170 evaluable patients.

Thus, a one-sided p-value ≦0.1 in the Chi-square test comparing PFS rates allows the trial to proceed without going to Step 2, the early survival futility assessment.

Assuming the PFS rate at 14 weeks is 50% in the control arm (as expected from reports on median PFS of about 3 months in patients treated with docetaxel in the 2nd line setting), the 10% significance level criterion translates into a critical absolute difference of 10%.

The power of the proposed rule to correctly detect a true benefit and enable study continuation is 87%, 80% and 76% for absolute true difference between arms of 18%, 16% and 15%, respectively, assuming control arm rate is 50%.

NOTE: The binary (PFS) assessment at the ˜14 week time point was chosen as opposed to time-to-event PFS analysis because in-clinic treatment visits schedule is more frequent for the experimental arm (every week) in comparison to the docetaxel control arm (every 3 weeks), whereas the scheduled assessment for progression (up to week 14) is only at two time points (8 weeks and 14 weeks) such that there is the possibility for unscheduled weekly progression assessment for the treatment arm and not for the control arm (leading to bias in time to event).

### Step 2: Overall Survival at 100 Events (Time-to-Event Analysis) (Only Analyzed if Step 1 Show One-Sided P-Value>0.1 for Difference in Rate)

The statistical rule was chosen to provide 28% false-positive probability, if in fact there is no difference in OS between the two arms, using 100 death events, thus leading to 72% probability to correctly stop the trial if indeed futile. The corresponding critical HR for declaring futility is HR=0.890.

Thus, a futility criteria with failure defined as having an observed HR of ≧0.890 in the OS analysis, or equivalently, trial is allowed to continue if observed HR<0.890.

The table below presents the estimated probability to continue the trial using the proposed futility rule, for various specifications of the true hazard ratio. As can be seen, if the true HR=0.75, then the probability of showing futility incorrectly would be 20%, and it would be as high as 30% if true HR=0.8.

| | Probability to | |
|---|---|---|
| True HR | continue (stop) | |
| 0.75 | 80% | (20%) |
| 0.77 | 76% | (24%) |
| 0.8 | | (30%) |
| 1 | 28% | (72%) |

### 2. Operating Characteristics of 2nd Interim Assessment for Futility

The second futility analysis may occur when 50% of the death events have occur (425 events) when approximately 800 patients are enrolled into the trial, at about 39-40 months after beginning of randomization. The stopping boundary was calculated to have 1% chance of falsely stopping the trial for futility if true HR=0.8, and provides 49% chance to correctly stop the trial if true HR=1. The corresponding critical HR is 1.0025.

### 3. Analysis Methods in the 1st Interim Assessment

### Binary PFS Variable Analysis

Analysis are based on 170 evaluable patients, defined as having a measurable disease at baseline (eligibility criteria into the study) and received at least one dose of study treatment. For each patient, a Week 14 “Alive Without Disease Progression” (AWP) status variable is defined as one (1) if the patient is alive and free of evidence of radiological disease progression at Week 14 assessment and zero (0) if otherwise. The proportions of patients in each arm for which AWP=1 are compared using Chi square test. Supportive analysis adjusting for imbalance of prognostic variables are performed using logistic regression. The assessment of progression for this endpoint are based on the results of the blinded independent central review of the Central Imaging Lab. If this criterion is not met and required improvement in PFS rate is not observed, then the early OS futility analysis is warranted to determine if the trial should be stopped.

### Early OS Futility Analysis at 100 Death Events:

All patients randomized up to realization of 100 death events are included in the analysis. A patient that is not reported as dead before data cut-off or has dropped out from survival follow-up are censored at his last known alive date. The differences in OS distribution between the two arms is summarized using the HR, as estimated from a Cox proportional hazards model (un-stratified). Supportive analysis adjusting for imbalance in important prognostic variables is performed by including such covariates into the model.

TABLE 2
Study Task Flow Chart

| | | | Treatment Period | | | | | | | Follow Up Periods | |
|---|---|---|---|---|---|---|---|---|---|---|---|
| | | | | | | | | | End of | Off | |
| | | | | | | | | | Treatment | Treatment | Survival |
| | | Up to | (Arm A only) | | | Cycle 1+ ^{b, g} | | | Visit | Follow-up | Follow-up |
| | | 28 days | Days −9 to −1 ^{a} | | | (±1 day for each | | | 28 ± 7 days | Every | Every |
| | | Screening | Min 5 days, Max 9 days | | | visit day) | | | from last | 4 weeks | 12 weeks |
| Procedure | | Visit | Dose 1 | Dose 2 | Dose 3 | Day 1 | Day 8 | Day 15 | dose | (±1 week) | (±1 week) |
| Informed consent | | • | | | | | | | | | |
| Demographic data | | • | | | | | | | | | |
| Disease/medical and smoking history ^{c} | | • | | | | | | | | | |
| Physical exam and weight ^{d} | | • | | | | • | | | • | | |
| Vital signs ^{e} | | • | • | • | • | • | | | • | | |
| Performance Status (ECOG) | | • | | | | • | | | • | | |
| FACT-L Questionnaire ^{f} | | • | | | | • | | | • | | |
| CT/MRI scan ^{g} | | • | | | | • ^{g} | | | • ^{g} | • ^{g} | |
| Electrocardiogram (ECG) ^{h} | | • | | | | | | | • | | |
| Hematology/chemistry ⁱ | | • | • ^{j} | | | • | • | | • | | |
| Urine dipstick (protein/blood) | | • | • ^{j} | | | • | | | • | | |
| Serum pregnancy test (β-hCG) ^{k} | | • | | | | | | | | | |
| Blood sample for serum Clusterin | | • | • | | | • | | | • | | |
| PK samples (custirsen) | | | • | | | • ^{l} | | | | | |
| Arm A | Ibuprofen/ | | • | • | • | | | | | | |
| patients | acetaminophen ^{m} | | | | | | | | | | |
| only | Custirsen infusions | | • | • | • | • | • | • | | | |

| | | | | | | | | | |
|---|---|---|---|---|---|---|---|---|---|
| Pre-medication prior to chemotherapy | | | | | • | | | | |
| Docetaxel infusion | | | | | • | | | | |
| AE recording ⁿ | • | • | • | • | • | • | • | • | |
| Concomitant medications | • | • | | | • | • | • | • | |
| Survival status | | | | | | | | | • |

^{a} Arm A patients only: During the Loading Dose Period, 3 custirsen infusions must be given between Days −9 and −1. The first dose must be given within 4 days after randomization. Each administration of custirsen between Day −9 and Day −1 must be a minimum of one day apart. There must be at least 1 non-infusion day (Day 0) and no more than 4 days between the last loading dose and Day 1 of Cycle 1.
^{b} For Arm B patients, Day 1 of Cycle 1 must be given within 4 days after randomization. For all patients, treatment should continue until disease progression, unacceptable toxicity, withdrawal of consent or investigator decision to stop treatment.
^{c} Collect EGFR and KRAS mutation status results, if available.
^{d} Height is measured only at screening visit. Subsequent physical examinations are abbreviated (i.e., limited to weight and assessing signs and symptoms of disease or toxicity)
^{e} Vital signs include blood pressure, heart rate and temperature. Arm A patients only: Vital signs are performed before and after completion of custirsen infusions during the custirsen Loading Dose Period and on Day 1 of each cycle. Vital signs should also be taken with any signs or symptoms (e.g., flushing, chills) during or immediately after an infusion. For all patients: Vital signs are performed at Screening, Day 1 of each cycle prior to study treatment, at the End of Treatment visit.
^{f} To be completed by the patient upon arrival at the clinic and before any study procedures or testing are performed.
^{g} All patients undergo CT or MRI scans of the chest and upper abdomen, as well as any other areas clinically indicated, at screening, then every 6 weeks (±one week), starting at week 8 after randomization (regardless of the treatment schedule) until disease progression. Note: CT scans are preferred; however, MRIs can be used for disease assessment as long as they are consistently performed for an individual patient's assessments. A chest and upper abdomen CT scan (MRI, if appropriate) that is performed as standard of care prior to consent for this study may be used as the screening test if obtained within 28 days prior to randomization and if accessible to the same facility where subsequent scans are performed and if it is of adequate quality for subsequent evaluations, according to the requirements of the central imaging center.
^{h} EGG is performed for all patients at Screening and End of Treatment Visit. ECG can be repeated at any visit if there is a clinical indication for an ECG.
ⁱ Hematology [White Blood Cell (WBC) count, hemoglobin, platelet count, absolute neutrophil and lymphocyte counts] and Chemistry [albumin, serum Creatinine, sodium, potassium, calcium, phosphorus, alkaline phosphatase, LDH, bilirubin (total and direct), SGOT (AST), and SGPT (ALT)] is drawn at screening, prior to the first loading dose custirsen infusion (unless screening blood draw was collected within 14 days of randomization), prior to infusion on Day 1 of each cycle and at the End-of-Treatment Visit. The hematology and serum chemistry laboratory tests can be performed up to 72 hours prior to the infusion on Day 1 of each cycle and is available before the start of treatment on those days.
^{j} Does not have to be repeated if screening sample was collected within 14 days prior to randomization.
^{k} For women of childbearing potential. Test is completed within 72 hours prior to randomization.
^{l} PK sampling (custirsen) is performed in a subset of patients in Arm A at the following 6 timepoints; before the first loading dose, on day Day 1 of Cycle 1 (end of custirsen infusion), and on Day 1 of Cycle 3 (predose, end of custirsen infusion and end of docetaxel infusion) and on Day 8 of Cycle 8 (predose).
^{m} For Arm A patients only, Ibuprofen (400 mg) or acetaminophen (paracetamol) (500-1000 mg) is given 30-60 minutes prior to and every 4-6 hours for 24 hours following each infusion of custirsen during the Loading Dose Period. Further administration of premedication following the Loading Dose Period is at the discretion of the Investigator.
ⁿ Adverse events to be graded using NCI CTCAE Version 4.0 and reported for each loading dose and at every cycle on Day 1 prior to treatment. The adverse event reporting period begins from the signing of informed consent and ends 28 days following the last dose of study treatment. Serious adverse events and grade 3 or higher adverse events that are ongoing at the End of Treatment visit is followed until each event resolves or is assessed as chronic.

After discontinuation of study treatment, all patients must be contacted every 12 weeks to collect further anticancer treatment and survival information.

### Results

The combination treatment of custirsen and docetaxel administered to arm A subjects is safe and well tolerated, with an acceptable adverse events profile. Arm A subjects (custirsen+docetaxel) have prolonged survival compared to Arm B subjects (docetaxel). Additionally, progression free survival is increased in Arm A subjects and a statistically significant higher proportion of Arm A subjects survive free of progression for at least 14 weeks compared to Arm B subjects. Overall progression free survival is improved in arm A subjects. One or more symptoms of NSCLC, such as chest pain, pleural effusions, pulmonary edema, dyspnea, or hemoptysis occasionally improve in Arm A subjects compared to Arm B subjects. Furthermore, quality of life improves in Arm A subjects compared to Arm B subjects.

### DISCUSSION

The experimental examples provided herein show that, when combined with custirsen, taxanes are particularly effective for the treatment of lung cancer, whether administered in the absence of additional chemotherapeutic agents or in combination with a platinum-based chemotherapeutic agent. Example 1 shows that the combination of custirsen with paclitaxel (a taxane) and carboplatin (a platinum-based chemotherapy agent) is effective at treating NSCLC of non-squamous histology, and Example 2 shows that the combination of custirsen with docetaxel is effective at treating NSCLC, even without an additional platinum-based chemotherapeutic agent. Therefore, it will be understood that the taxanes, and the combinations of taxanes and platinum-based chemotherapeutic agents disclosed herein will be particularly effective for treating lung cancer when combined with custirsen.

### REFERENCES

1. Calvert et al., Carboplatin dosage: prospective evaluation of a simple formula based on renal function. J Clin Oncol. 1989; 7:1748-1756.
2. Carboplatin Package Insert, Bedford Laboratories, 2010; Accessed from www.bedfordlabs.com/products/inserts/carboplatin_pi.pdf on May 4, 2011.
3. Cockcroft and Gault, Prediction of creatinine clearance from serum creatinine. Nephron 16; 1976, (1): 31-41.
4. D'Addario et al., Metastatic non-small-cell lung cancer: ESMO Clinical Practice Guidelines for diagnosis, treatment and follow-up. Annals of Oncology.
5. Fidias and Novello, Strategies for Prolonged Therapy in Patients with Advanced Non-Small-Cell Lung Cancer. Journal of Clinical Oncology, 2010, 28(34): 5116-5123.
6. Jemal et al., Global Cancer statistics, 2011. CA Cancer J Clin; 2011, 61:69-90.
7. Knox et al., Mechanism of cytotoxicity of anticancer platinum drugs: evidence that cis-diamminedichloroplatinum(II) and cis-diammine-(1,1-cyclobutanedicarboxylato)platinum(II) differ only in the kinetics of their interaction with DNA. Cancer Research, 1986, 46(4 Pt 2):1972-9.
8. Kuriyama, Effect of taxol on first and second meiotic spindle formation in oocytes of the surf clam, Spisula solidissima. J Cell Sci. 1986 84: 153-64.
9. Langer et al., The Evolving Role of Histology in the Management of Advanced Non-Small-Cell Lung Cancer. Journal of Clinical Oncology, 2010, 28(36):5311-5320.
10. National Comprehensive Cancer Network Clinical Practice Guidelines in Oncology, Non-Small Cell Lung Cancer, V.2.2010. National Comprehensive Cancer Network, Inc., Mar. 5, 2010.
11. National Cancer Institute, General Information about Non-Small Cell Lung Cancer. http://www.cancer.gov/CANCERTOPICS/PDQ/TREATMENT/NON-SMALL-CELL-LUNG/PATIENT, accessed Feb. 24, 2011.
12. OncoGenex Technologies, A Study of OGX-011/Gemcitabine/Platinum-Based Regimen in Stage IIIB/IV Non-Small Cell Lung Cancer (NSCLC). 2005, accessed from www.clinical trials.gov/ct2/showNCT00138658, accessed Feb. 24, 2011.
13. Pirker et al., Cetuximab plus chemotherapy in patients with advanced non-small-cell lung cancer (FLEX): An open-label randomized phase III trial. Lancet, 2009, 373:1525-1531.
14. Rowinsky et al., Taxol: A Novel Investigational Antimicrotubule Agent. J. Natl Cancer Inst, 1990, 82(15):1247-59.
15. Sandler et al., Paclitaxel-carboplatin alone or with bevacizumab for non-small-cell lung cancer. N Eng J Med, 2006, 355:2542-2550.
16. Soon et al., Duration of chemotherapy for advanced non-small-cell-lung cancer: A systematic review and meta-analysis of randomized clinical trials.
17. Taxol Package Insert, Bristol-Myers Squibb Company, 2010; Accessed from http://packageinserts.bms.com/pi/pi_taxol.pdf on May 5, 2011.
18. Teicher et al., Influence of Schedule on Alkylating Agent Cytotoxicity in Vitro and in Vivo. Cancer Research, 1989, 49:5994-5998.

What is claimed is:

1. A method of treating a human patient afflicted with unresectable, advanced or metastatic non-small cell lung cancer comprising periodically administering to the human patient chemotherapy comprising an amount of a taxane, and 640 mg of an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, thereby treating the human patient afflicted with unresectable, advanced or metastatic non-small cell lung cancer.

2. The method of claim 1, wherein the taxane is paclitaxel.

3-4. (canceled)

5. The method of claim 2, wherein during the chemotherapy the paclitaxel is administered to the human patient on the first day of each of up to six three-week chemotherapy cycles.

6. (canceled)

7. The method of claim 1, wherein the taxane is docetaxel, baccatin III, baccatin V, taxol B (cephalomannine), taxol C, taxol D, taxol E, taxol F, taxol G, cabazitaxel, larotaxel, ortataxel (14 beta-hydroxydeacetyl baccatin III), tesetaxol, 10-deacetyl baccatin III, 7-xylosyl-10-deacetyl cephalomannine, 7-xylosyl-10-deacetyl paclitaxel, 10-deacetyl cephalomannine, 7-xylosyl-10-deacetyl taxol C, 10-deacetyl paclitaxel, 7-xylosyl paclitaxel, 10-deacetyl taxol C, 10-deacetyl-7-epi cephalomaunine, 7-xylosyl taxol C, 10-deacetyl-7-epipaclitaxel, 7-epi cephalomaunine, 7-epi paclitaxel, 7-O-methylthiomethyl paclitaxel, 7-deoxy docetaxel, taxanime M, PG-paclitaxel, or DHA-paclitaxel.

8. The method of claim 1, wherein the taxane is docetaxel.

9-12. (canceled)

13. The method of claim 2, wherein the chemotherapy further comprises an amount of a platinum-based chemotherapeutic agent.

14. (canceled)

15. The method of claim 13, wherein the platinum-based chemotherapeutic agent is carboplatin or cisplatin.

16-17. (canceled)

18. The method of claim 15, wherein during the chemotherapy the carboplatin is administered to the human patient on the first day of each of up to six three-week chemotherapy cycles.

19-22. (canceled)

23. The method of claim 1, wherein the non-small cell lung cancer is stage IV lung cancer.

24. The method of claim 1, wherein the non-small cell lung cancer is of non-squamous histology.

25. The method of claim 1, wherein the treating includes prolonging survival of the human patient.

26. The method of claim 1, wherein the treating includes prolonging survival of the human patient which prolonged survival is free of progression of the non-small lung cancer.

27. The method of claim 26, wherein the human patient survives free of progression of the non-small cell lung cancer for at least 14 weeks.

28. (canceled)

29. The method of claim 1, wherein the non-small cell lung cancer is lung adenocarcinoma or lung large cell carcinoma.

30-32. (canceled)

33. The method of claim 1, wherein the human patient has not received treatment for non-small cell lung cancer for at least 1 year.

34. (canceled)

35. The method of claim 1, wherein the human patient has before initiation of the periodic administration received a chemotherapeutic agent for the treatment of lung cancer.

36. (canceled)

37. The method of claim 1, further comprising the steps of:
i) measuring the level of serum clusterin present in the blood of the human patient prior to the administration of the anti-clusterin oligonucleotide;
ii) determining whether the level of serum clusterin present in the human patient is lower than a predetermined upper threshold level of baseline serum clusterin below which a human patient is likely to substantially benefit from anti-clusterin therapy; and
iii) administering the anti-clusterin oligonucleotide only if the level of serum clusterin present in the blood of the human patient is lower than the predetermined upper threshold level of baseline serum clusterin.

38-39. (canceled)

40. The method of claim 1, further comprising the steps of:
iv) administering to the human patient the anti-clusterin oligonucleotide in an initial dosage and treatment protocol;
v) thereafter testing the human patient to determine a level of serum clusterin after a period of treatment with the anti-clusterin oligonucleotide intended to reduce clusterin expression;
vi) determining an adjusted dosage and treatment protocol based on the determined level of serum clusterin; and
vii) administering to the human patient the anti-clusterin oligonucleotide in accordance with the adjusted dosage and treatment protocol.

41-43. (canceled)

44. A composition or combination for treating a human patient afflicted with unresectable, advanced or metastatic non-small cell lung cancer, comprising chemotherapy comprising a taxane and, optionally, a platinum-based chemotherapeutic agent, and an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19.

45-48. (canceled)

49. A package for use in the treatment of a human patient afflicted with unresectable, advanced or metastatic non-small cell lung cancer, comprising chemotherapy comprising a taxane and, optionally, a platinum-based chemotherapeutic agent, and an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′ deoxynucleotides, and has 5-methylcytosines at nucleotides 1, 4, and 19, and instructions for the use of the chemotherapy in combination with the anti-clusterin oligonucleotide for the treatment of unresectable, advanced or metastatic non-small cell lung cancer.

50-114. (canceled)
